(19) United States
(12) Patent Application Publication
(10) Pub. No.: US 2015/0297213 A1
(43) Pub. Date: Oct. 22, 2015

(54) FIXATION ASSEMBLY AND RELATED METHODS OF USE AND MANUFACTURE

(71) Applicant: Boston Scientific Scimed, Inc., Maple Grove, MN (US)

(72) Inventors: Laurie A. Lehtinen, Nashua, NH (US); Milton G. Azevedo, Marlborough, MA (US); Matthew R. Jagelski, West Roxbury, MA (US); John B. Golden, Norton, MA (US)

(21) Appl. No.: 14/687,192

(22) Filed: Apr. 15, 2015

Related U.S. Application Data
(60) Provisional application No. 61/980,224, filed on Apr. 16, 2014.

(51) Int. Cl. A61B 17/04

(52) U.S. Cl. CPC A61B 17/0401; A61B 2017/0409; A61B 2017/00818

(57) ABSTRACT

A fixation assembly and associated methods for making and using the assembly. The assembly includes a hub assembly having a needle hub secured to a needle and a stylet hub secured to a stylet extending through a lumen of the needle. The stylet hub is selectively actuatable relative to the needle hub between a first position and a second position. A stop is removably coupled to the hub assembly when the stylet hub is in the first position to prevent actuation of the stylet hub to the second position. The stop is removed from the hub assembly to allow actuation of the stylet hub to the second position.

### CROSS-REFERENCE TO RELATED APPLICATIONS

This application claims priority to U.S. Provisional Application No. 61/980,224, filed Apr. 16, 2014, the entire disclosure of which is incorporated herein by reference.

### TECHNICAL FIELD

Some embodiments relate to devices and assemblies for percutaneous fixation of organs, such as a stomach, and methods for manufacturing and using the same. More particularly, some embodiments of the present disclosure relate to assemblies and methods for securing the abdominal wall to the gastric wall, such as during a gastropexy procedure.

### BACKGROUND

Gastropexy is a procedure that may secure the gastric wall to the abdominal wall during placement of a feeding tube (e.g., a gastric, jejunal or transgastric jejunal feeding tube) inside a patient's stomach or jejunum. The procedure generally employs a fixation assembly having a needle that can be entered into the patient's stomach percutaneously. Once the needle reaches the patient's stomach, a fastener, such as a T-shaped fastener, coupled to a suture is deployed inside the stomach. Further, the suture can be pulled proximally to draw the stomach toward the abdominal wall and tightly secure the stomach with the abdominal wall using the fastener and an accompanying suture anchor secured to the suture on the external surface of the abdomen. The attachment initially allows for ease of placement of the feeding tube, as well as dilation of the stoma tract. Attaching the stomach to the abdominal wall may allow adhesions to form between the two tissues so that the attachment remains after the sutures are absorbed or removed. Attaching the stomach to the abdominal wall with gastropexy may help to prevent inadvertent displacement of the feeding tube, or migration of the feeding tube into the peritoneal cavity. Migration of the feeding tube into the peritoneal cavity may lead to exposure of contamination leading to health risks for the patient. For example, nutrition administered through a feeding tube while it is positioned in the peritoneal space may lead to significant health risks.

Accordingly, it may be desirable to provide a percutaneous fixation assembly for a percutaneous fixation that can be handled more easily and provides accurate deployment of the fastener inside the patient's stomach while preventing premature deployment of the fastener.

### SUMMARY

One illustrative embodiment includes a percutaneous fixation assembly. The assembly includes a hub assembly having a needle hub and a stylet hub selectively actuatable relative to the needle hub between a first position and a second position. The assembly further includes an elongate needle, a stylet, and a stop. The elongate needle has a proximal end secured to the needle hub, and a lumen extending between the proximal end and a distal end. The stylet is secured to the stylet hub and extends into a lumen of the elongate needle. The stop is removably coupleable to the hub assembly, such that the stop is coupled to the hub assembly in the first position to impede or prevent actuation of the stylet hub to the second position, where the stop is removable from the hub assembly to allow actuation of the stylet hub to the second position.

Another illustrative embodiment includes a method of manipulating a percutaneous fixation assembly. The method includes operating a percutaneous needle assembly, where the assembly includes a hub assembly, an elongate needle, and a stylet as disclosed above. The assembly further includes a suture anchor which may be used as the stop of the above discussed embodiment. The method includes removing the suture anchor from the hub assembly to allow actuation of the stylet hub to the second position, and actuating the stylet hub to the second position after removing the suture anchor from the hub assembly.

Another illustrative embodiment includes a gastropexy assembly having a hub assembly, an elongate needle, a stylet, a suture anchor, and a fastener. The hub assembly includes a needle hub and a stylet hub selectively actuatable relative to the needle hub between a first position and a second position. The elongate needle has a proximal end secured to the needle hub and a lumen extending between the proximal end and a distal end. The stylet is secured to the stylet hub while extending into the lumen of the elongate needle. Further, the suture anchor is removably coupleable to the hub assembly when the stylet hub is in the first position, thereby impeding or preventing actuation of the stylet hub to the second position. The fastener is positionable in the lumen of the elongate needle of the stylet having a suture extending from the fastener that passes through an opening in the suture anchor. The suture anchor is removable from the hub assembly to allow actuation of the stylet hub to the second position.

Yet another embodiment is directed to a method of manipulating a percutaneous fixation assembly. The method includes operating a percutaneous fixation assembly as discussed above. The method further includes removing the suture anchor from the hub assembly to allow actuation of the stylet hub to the second position, and actuating the stylet hub to the second position after removing the suture anchor from the hub assembly.

The above summary of some embodiments is not intended to describe each disclosed embodiment or every implementation of the present disclosure. The Figures, and Detailed Description, which follow, more particularly exemplify these embodiments, but are also intended as exemplary and not limiting.

### BRIEF DESCRIPTION OF THE DRAWINGS

The disclosure may be more completely understood in consideration of the following detailed description of various embodiments in connection with the accompanying drawings, in which:

FIG. 1 is a perspective view of an exemplary percutaneous fixation assembly, in accordance with some embodiments of the present disclosure;

FIG. 1A is a longitudinal cross-sectional view of the exemplary percutaneous fixation assembly of FIG. 1 illustrating a stylet hub in a first position;

FIG. 2 is a perspective view of the percutaneous fixation assembly of FIG. 1 illustrating a suture anchor removed from the hub assembly;

FIGS. 3 and 3A are perspective and longitudinal cross-sectional views, respectively, of the percutaneous fixation assembly of FIG. 1 illustrating the stylet hub in a second position;

FIG. 4 is a perspective view of an exemplary suture anchor for use with the percutaneous fixation assembly of FIG. 1;

FIG. 5 is a side view of the suture anchor of FIG. 4;

FIG. 6 is a cross-sectional top view of the suture anchor of FIG. 4 in an unlocked state;

FIG. 7 is another cross-sectional top-view of the suture anchor of FIG. 4 in a locked state;

FIG. 8 is a perspective view of another exemplary suture anchor for use with the percutaneous fixation assembly of FIG. 1;

FIG. 9 is a perspective view of another exemplary percutaneous fixation assembly, in accordance with some embodiments of the present disclosure;

FIG. 10 is a perspective view showing various components of the percutaneous fixation assembly of FIG. 9;

FIG. 11 is a longitudinal cross-sectional view of the percutaneous fixation assembly of FIG. 9 illustrating the stylet hub in a first position;

FIG. 12 is a longitudinal cross-sectional view of the percutaneous fixation assembly of FIG. 9 illustrating the stylet hub in an intermediate position;

FIG. 13 is a longitudinal cross-sectional view of the percutaneous fixation assembly of FIG. 9 illustrating the stylet hub in a second position;

FIG. 14 is a perspective view of yet another exemplary percutaneous fixation assembly, in accordance with some embodiments of the present disclosure;

FIG. 14A is a longitudinal cross-sectional view of the percutaneous fixation assembly of FIG. 14 illustrating the stylet hub in a first position;

FIG. 15 is a perspective view of the percutaneous fixation assembly of FIG. 14 having a clip removed from the hub assembly;

FIG. 16 is a perspective view of the percutaneous fixation assembly of FIG. 14 illustrating the stylet hub in a second position;

FIG. 16A is a longitudinal cross-sectional view of the percutaneous fixation assembly of FIG. 14 illustrating the stylet hub in a second position;

FIGS. 17A-17E illustrate an exemplary method for using the percutaneous fixation assembly of FIG. 1;

FIG. 18 is a perspective view of an abdominal wall secured to a gastric wall;

FIG. 19 is a perspective view of another exemplary suture anchor for use with a percutaneous fixation assembly of this disclosure;

FIG. 20 is a side view of the suture anchor of FIG. 19;

FIG. 21 is a cross-sectional top view of the suture anchor of FIG. 19 in an unlocked state;

FIG. 22 is another cross-sectional top-view of the suture anchor of FIG. 19 in a locked state;

FIG. 23 is a perspective view of another exemplary suture anchor for use with a percutaneous fixation assembly of this disclosure;

FIG. 24 is a side view of the suture anchor of FIG. 23;

FIG. 25 is a cross-sectional top-view of the suture anchor of FIG. 23 in a locked state;

FIG. 26 is an exploded view of another exemplary suture anchor for use with a percutaneous fixation assembly of this disclosure;

FIG. 27 is a cross-sectional top view of the suture anchor of FIG. 26 in an unlocked state;

FIG. 28 is another cross-sectional top-view of the suture anchor of FIG. 26 in a locked state;

FIG. 29 is a top view of the suture anchor of FIG. 26 in the locked state;

FIG. 30 is a perspective view of the suture anchor of FIG. 26 illustrating the use of an instrument to unlock the suture anchor; and

FIG. 31 is a cross-sectional top view of the suture anchor of FIG. 26 in which an instrument is used to unlock the suture anchor.

### DETAILED DESCRIPTION

Definitions of certain terms are provided below and shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same or substantially the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a,” “an,” and “the” include or otherwise refer to singular as well as plural referents, unless the content clearly dictates otherwise. As used in this specification and the appended claims, the term “or” is generally employed to include “and/or,” unless the content clearly dictates otherwise.

The following detailed description should be read with reference to the drawings, in which similar elements in different drawings are identified with the same reference numbers. The drawings, which are not necessarily to scale, depict illustrative embodiments and are not intended to limit the scope of the disclosure.

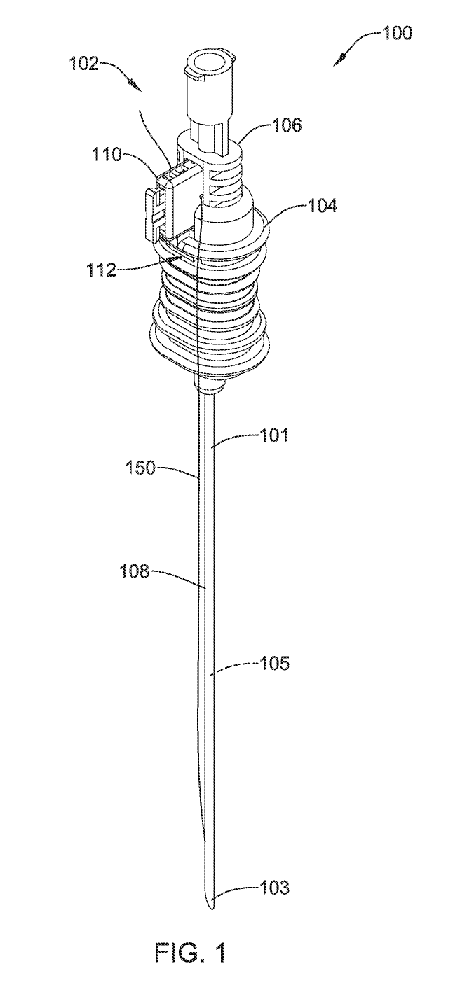

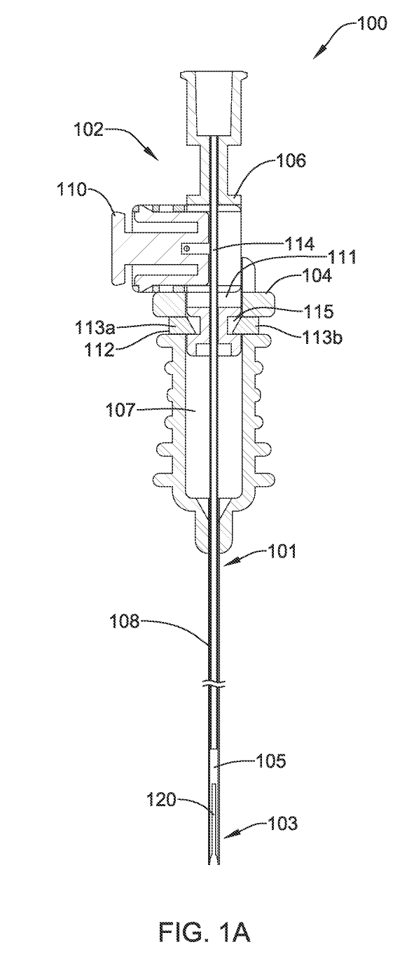

FIGS. 1-1A are perspective and longitudinal cross-sectional views of an exemplary percutaneous fixation assembly 100, in accordance with some embodiments of the present disclosure.

The percutaneous fixation assembly 100 may include a hub assembly 102 having a needle hub 104 and a stylet hub 106. As shown, in FIG. 1A, the needle hub 104 may include an inner surface defining a bore 107 such that a distal portion of the stylet hub 106 may extend into the bore 107 of the needle hub 104 and move longitudinally relative to the needle hub 104.

The fixation assembly 100 may further include a retention mechanism configured to resist relative movement between the needle hub 104 and the stylet hub 106 unless a force greater than a threshold amount of force is applied. For example, the retention mechanism may include a clip, such as a C-shaped clip 112 having tabs 113a and 113b. The clip 112 may be mounted on the needle hub 104 and partially surround the needle hub 104 such that the two tabs 113a and 113b may extend into the bore 107 of the needle hub 104 from opposing sides of the bore 107. To this end, the needle hub 104 may include a channel or groove for receiving the C-shaped clip 112, and may include openings on opposite sides of the needle hub 104 for receiving the tabs 113a, 113b therein, such that the tabs 113a and 113b may pass therethrough to extend into the bore 107. A distal portion of the stylet hub 106 may include a recess 115 (e.g., groove, hole, opening, rim, etc.) formed on its external surface such that the tabs 113a and 113b may be positioned into the recess 115, thereby mechanically coupling the stylet hub 106 and the needle hub 104 in a first position.

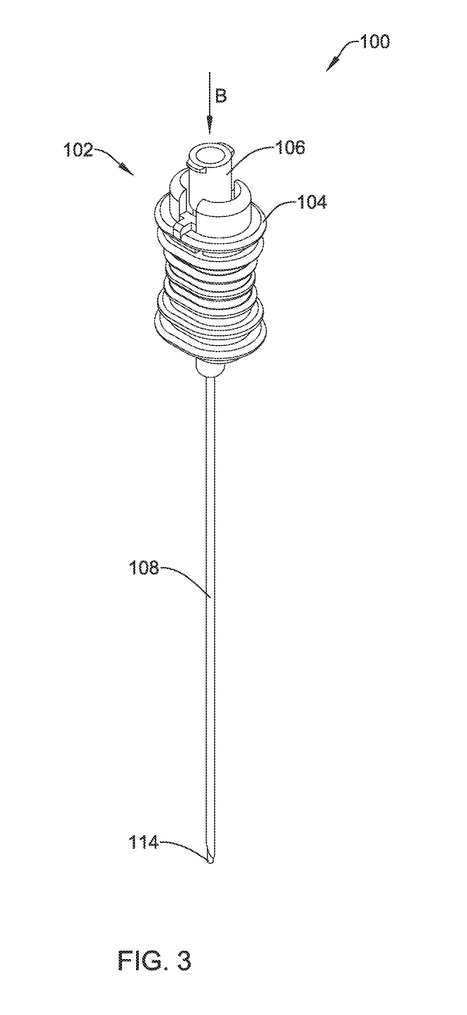

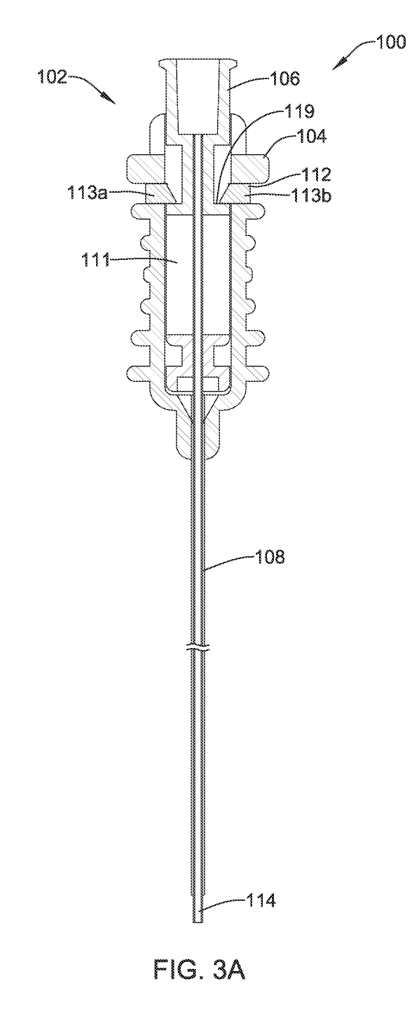

The stylet hub 106 may be actuated relative to the needle hub 104. For example, the stylet hub 106 may be actuated relative to the needle hub 104 along a longitudinal axis of the elongate needle 108 and stylet 114 to move the stylet 114 through the elongate needle 108. For example, a push force may be applied in a distal direction along the longitudinal axis as shown by arrow B in FIG. 3 to actuate the stylet hub 106 from a first position (shown in FIGS. 1 and 1A) to a second position (shown in FIGS. 3 and 3A). The details of actuation of the stylet hub 106 and the first and second positions will be discussed in detail below.

The fixation assembly 100 may also include an elongate needle 108 having a proximal end 101, a distal end 103, and a lumen 105 extending between the proximal and distal ends 101 and 103. The proximal end 101 of the elongate needle 108 may be secured to the needle hub 104. Various securing mechanisms such as, but not limited to, molding, gluing, threading, snap-fitting, and so forth may be employed to secure the elongate needle 108 to the needle hub 104. The elongate needle 108 may be introduced into a patient's body via a sharpened distal tip at its distal end 103. In an example, the elongate needle 108 may pierce through the skin of the patient to be inserted into the stomach and/or gastric cavity of the patient to secure the stomach to the abdominal wall during placement of feeding tubing. Such an elongate needle 108 may have a sharp distal tip and a length suitable for travelling a path from external skin to an internal gastric cavity of the patient. In addition, the elongate needle 108 may have an outer diameter suitable for penetrating through skin and to traverse through the flesh of the patient, such as to cross the wall of a desired organ (e.g., the stomach) minimizing or preventing any injury to the surrounding tissue. An exemplary elongate needle 108 may include a 12 gauge, 14 gauge, or 16 gauge needle, for example.

The fixation assembly 100 may further include a stylet 114 (as shown in FIG. 1A) slidably positioned in the lumen 105 of the elongate needle 108. The stylet 114 may be secured to the stylet hub 106 using any suitable securing mechanisms such as, but not limited to, molding, gluing, threading, snap-fitting, and so forth. The stylet 114 may have a proximal end secured to the stylet hub 106 and a distal end extending into the lumen 105 of the needle hub 104. In some instances, the stylet 114 may be hollow and include a lumen extending between the proximal and distal ends of the stylet 114. In other instances, the stylet 114 may be a solid stylet devoid of a lumen. The lumen of the stylet 114 may be in fluid communication with a proximal port, such as a luer fitting, of the stylet hub 106 for passing fluid (e.g., air, carbon dioxide, saline, contrast fluid, etc.) therethrough during a medical procedure. For example, in a gastropexy procedure, if the elongate needle 108 is introduced into the patient's stomach for placing a feeding tube, then a physician may need to confirm an accurate placement of the distal end 103 of the elongate needle 108 prior to deployment of the fastener 152 as discussed herein. In such an instance, a syringe may be attached to the stylet hub 106 to aspirate air or other fluid from the patient's stomach through the lumen of the stylet 114. Observation of aspirated air or other fluid in the syringe may confirm the accurate positioning of the distal end 103 of the elongate needle 108 inside the stomach cavity. For example, the stomach may be insufflated with air or carbon dioxide during the procedure, which may be aspirated in the syringe to confirm accurate placement of the distal end 103 of the needle 108 inside the stomach cavity. Alternatively, a contrast media may be injected through a syringe attached to the stylet hub 106 through the lumen of the stylet 114 to confirm accurate placement of the needle 108 by visualizing the location of the injected contrast media using fluoroscopic imaging. Upon confirmation of accurate placement of the distal end 103 of the elongate needle 108 in the stomach, the physician may deploy the fastener from the elongate needle 108.

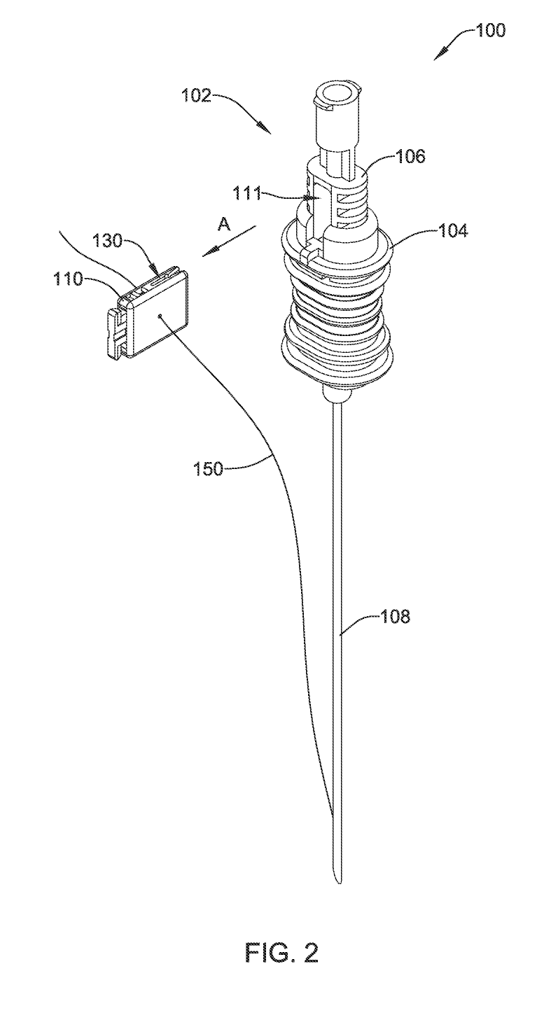

The fixation assembly 100 may also include a stop, which may be configured to be removably coupled to the hub assembly 102. In an example, the stop includes a suture anchor 110 that may be coupled to the hub assembly 102 to prevent actuation of the stylet hub 106 from the first position to the second position. For example, the suture anchor 110 may be positioned in an opening 111 (as shown in FIG. 2) formed in the stylet hub 106. The opening 111 may be made along a central longitudinal axis of the stylet hub 106 having dimensions suitable for receiving the suture anchor 110. The suture anchor 110 may engage the needle hub 104 and the stylet hub 106 to restrain movement between the needle hub 104 and the stylet hub 106 when coupled to the hub assembly 102.

The suture anchor 110 may include an elongate channel 130 (see FIG. 2) that may extend substantially parallel to the longitudinal axis of the elongate needle 108 and stylet 114 when coupled to the hub assembly 102. The stylet 114 may be positioned in the elongate channel 130 with a first portion of the suture anchor 110 on a first side of the stylet 114 and a second portion of the suture anchor 110 on an opposite, second side of the stylet 114 as the suture anchor 110 is coupled to the stylet hub 106 (e.g. positioned in the opening 111). Thus, the stylet 114 may extend from the elongate channel 130 proximal of the suture anchor 110 and distal of the suture anchor 110 when the suture anchor 110 is positioned in the opening 111. In some instances, the suture anchor 110 may snap onto the stylet 114 when positioned in the opening 111.

Insertion of the suture anchor 110 in the opening 111 may maintain the stylet hub 106 in the first position. In this position, the suture anchor 110, acting as a stop, may impede or prevent the stylet hub 106 from being actuated relative to the needle hub 104 to the second position from the first position.

The suture anchor 110 positioned in the stylet hub 106 may prevent or restrict distal movement of the stylet hub 106 relative to the needle hub 104. Such a restriction of movement of the stylet hub 106 may maintain the stylet hub 106 in the first position, such that the distal end of the stylet 114 remains proximal to the sharpened distal tip at the distal end 103 of the elongate needle 108 while being within the lumen 105 of the elongate needle 108. Discussed in greater detail later, the stylet 114 may be configured to deploy a fastener (not shown) that may be disposed within the lumen 105 of the elongate needle 108 distal of the stylet 114. In the first position, the fastener may remain inside the lumen 105 of the elongate needle 108 distal of the distal end of the stylet 114 disposed therein. The fastener may be secured to a suture 150, described later herein, which may pass out of the elongate needle 108 through a slot 120 formed in a sidewall of the elongate needle 108 such that the suture 150 may extend along an exterior of the elongate needle 108 to the hub assembly 102 or the suture anchor 110 and pass through the suture opening in the suture anchor 110 while the suture anchor 110 is attached to the hub assembly 102.

In a gastropexy procedure, the percutaneous fixation assembly 100 may include a fastener secured to a suture that may be configured to secure a patient's stomach to the abdominal wall during placement of a feeding tube inside the stomach or jejunum. In a pre-deployed state, the fastener may be disposed within the lumen 105 of the elongate needle 108 located distal of the distal end of the stylet 114. The stylet hub 106 may be in the first position in the pre-deployed state of the fastener. Therefore, the suture anchor 110 coupled with the hub assembly 102 may impede or prevent any inadvertent deployment of the fastener. In other words, with the suture anchor 110 positioned in the opening 111 and acting as a stop, the stylet hub 106 may not appreciably move relative to the needle hub 104, and thus the stylet 114 may not push the fastener out of the lumen 105 of the needle 108. Thereby, the suture anchor 110 may provide a pre-deployment safety mechanism for the fastener in the gastropexy procedure.

The suture anchor 110, which may have the suture 150 passing through the suture opening of the suture anchor 110, may be removed from the stylet hub 106, as shown in FIG. 2, to allow actuation of the stylet hub 106 relative to the needle hub 104. For example, the suture anchor 110 may be removed from the hub assembly 102 by pulling the suture anchor 110 along a direction A (see arrow shown in FIG. 2). In some instances, the direction A may be generally perpendicular to the longitudinal axis of the elongate needle 108 and stylet 114. The stylet hub 106 may now be shifted to the second position, which is discussed now with respect to FIGS. 3 and 3A.

FIGS. 3 and 3A are perspective and longitudinal cross-sectional views, respectively, of the exemplary percutaneous fixation assembly 100 of FIG. 1 illustrating the stylet hub 106 in the second position. As shown in FIG. 3, the stylet hub 106 may be moved along direction B (see arrow) to actuate the stylet hub 106 to the second position. In some instances, direction B may be generally parallel to the longitudinal axis of the elongate needle 108 and stylet 114. In the second position, the distal portion of the stylet hub 106 may be pushed distally within the bore 107 of the needle hub 104. The push force may sufficient to disengage the tabs 113a and 113b positioned within the recesses 115. For example, the tabs 113a, 113b may include angled surfaces which engage surfaces of the stylet hub 106 such that distal movement of the stylet hub 106 causes the tabs 113a, 113b to deflect outward in opposite directions allowing the tabs 113a, 113b to disengage the recesses 115 and allow clearance of the stylet hub 106 past the tabs 113a, 113b. For instance, the tabs 113a, 113b may deflect outward in opposite directions generally perpendicular to the longitudinal axis of the elongate needle 108 and stylet 114. Further, pushing the stylet hub 106 may distally move the distal portion of the stylet hub 106 within the bore 107. As a result, the tabs 113a and 113b may deflect back inward above the rim 119 of the stylet hub 106 formed along a proximal portion of the stylet hub 106 when moved to the second position. Interference between the tabs 113a, 113b and the rim 119 (or other recessed portion of the stylet hub 106) may prevent the stylet hub 106 from returning to the first position from the second position.

In the second position, the distal tip, such as a blunt distal tip, of the stylet 114 may move distally past the distal end 103 of the elongate needle 108 and deploy the fastener into the gastric cavity of the patient from the lumen 105 of the needle 108, during the gastropexy procedure discussed above. The fastener having been disposed within the lumen 105 of the elongate needle 108 distal to the stylet 114 in the first position. In addition to the deployment of the fastener, the blunt distal tip of the stylet 114 may extend distally past the sharpened distal tip of the needle 108 to facilitate blunting of the elongate needle 108.

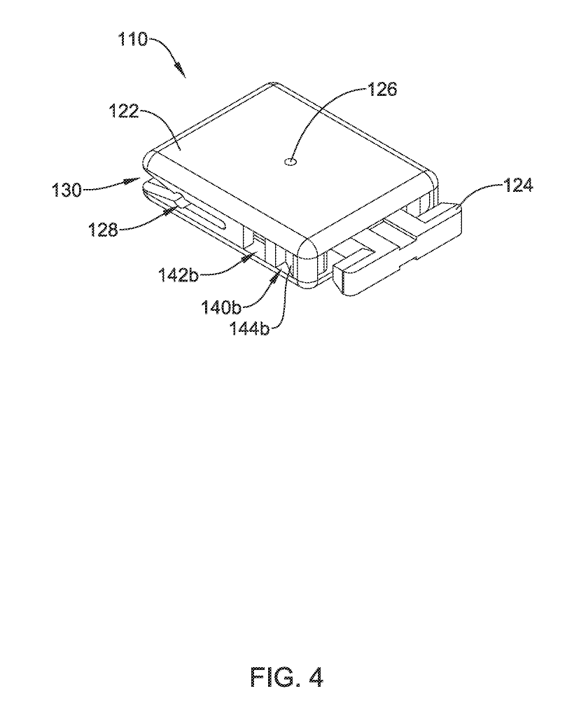

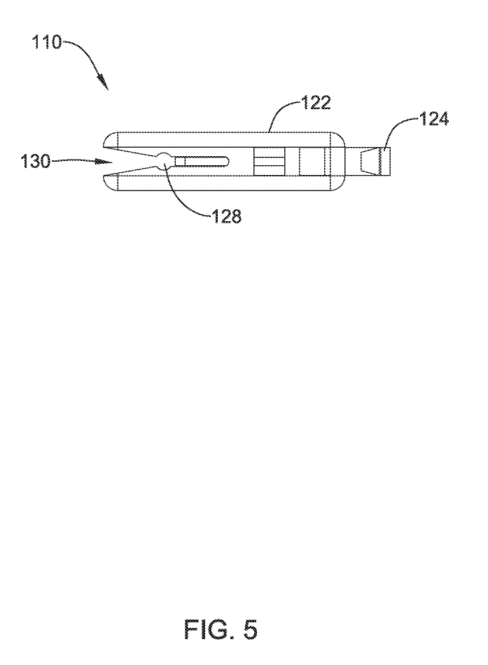

FIGS. 4-5 are perspective and side views of an exemplary suture anchor 110 for use with the percutaneous fixation assembly of FIG. 1. The suture anchor 110 may be configured to securely lock onto a suture for use in a medical procedure. Furthermore, the suture anchor 110 additionally functions as a stop when coupled to the handle assembly 102 of the percutaneous fixation assembly 100.

The suture anchor 110 may be formed of a unitary, single component construction, or the suture anchor 110 may be formed of multiple components. For example, as shown, the suture anchor 110 may be formed as a two-component structure, which may include a body 122 and a locking mechanism 124 movable relative to the body 122. The suture anchor 110 may have a rectangular configuration, where the locking mechanism 124 may be placed inside a cavity formed within the body 122. The locking mechanism 124 may be movable in the cavity of the body 122 between a first, unlocked position and a second, locked position. In the illustrated embodiment, the locking mechanism 124 may include a T-shaped structure that may be inserted into the cavity formed within the body 122. Although the suture anchor 110 is shown to be made as a two-component structure, the suture anchor 110 may be made as a unitary or monolithic structure, in which a locking mechanism portion is movable or deflectable relative to a body portion between an unlocked position and a locked position, if desired.

Further, as shown in FIG. 5, the suture anchor 110 may include an elongate channel 130 extending from one edge of the body 122 to an opposing edge of the body 122 that may receive the stylet 114 when the suture anchor 110 is positioned in the stylet hub 106. For example, the channel 130 may include an enlarged opening 128 in which the stylet 114 may be positioned when the suture anchor 110 is clipped onto or otherwise coupled to the stylet 114. The enlarged opening may have a diameter larger than the width of a portion of the channel 130 proximate thereto. For example, the width of the channel 130 may taper from a width greater than the diameter of the stylet 114 to a width less than the diameter of the stylet 114 proximate the enlarged opening 128. Accordingly, pushing the stylet 114 past the portion of the channel having a width less than the diameter of the stylet 114 and into the enlarged opening 128 may create a snap fit to retain the suture anchor 110 clipped onto the stylet 114.

The suture anchor 110 may also include a suture opening 126 configured to pass a suture therethrough. The suture opening 126 may be a through hole extending through the body 122 from a lower surface to an upper surface of the body 122, for example.

Material(s) employed to manufacture the suture anchor 110 may include any suitable biocompatible material, such as metals, polymers, composites, alloys, and so on. In some instances, the material of the body 122 may have rigidity relatively greater than that of the locking mechanism 124, to impede or prevent the locking mechanism 124 from deforming the body 122 as the two structures become locked. In some embodiments, the two structures may be made up of different materials. In some embodiments, the rigidity of the two structures of the suture anchor 110 may be the same, and the body 122 and locking mechanism 124 may be formed of the same materials.

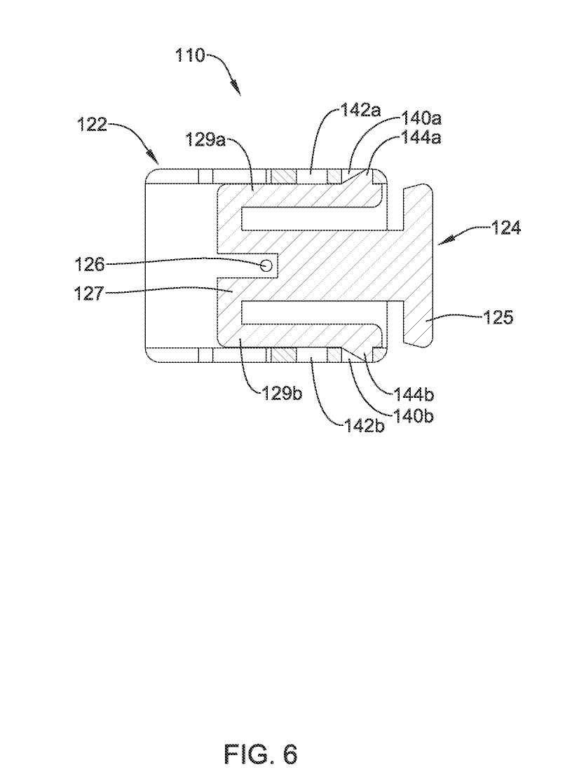

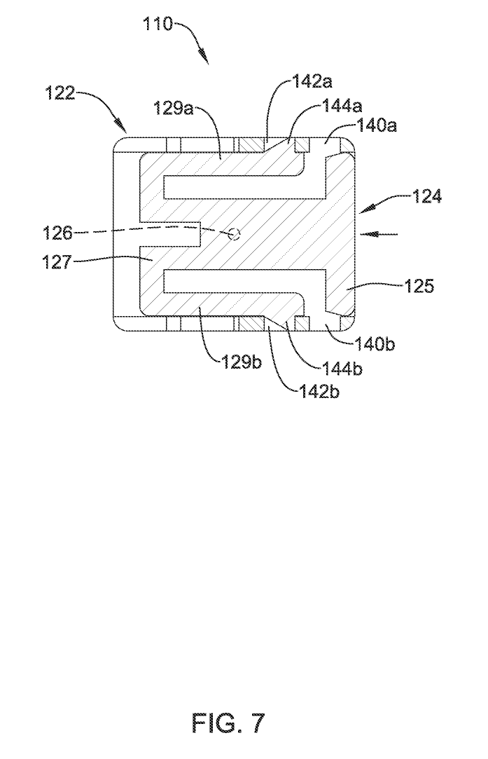

FIGS. 6-7 are cross-sectional top-views of the suture anchor 110 of FIG. 4 in unlocked and locked positions, respectively. As shown in FIG. 6, the locking mechanism 124 of the suture anchor 110 may have a T-shaped configuration that includes a proximal portion 125 and a distal portion 127. In the unlocked position, the proximal portion 125 may remain outside of the cavity of the body 122, whereas the distal portion 127 may be disposed within the cavity of the body 122. The distal portion 127 may be disposed substantially parallel along a longitudinal axis of the body 122 and actuatable along the longitudinal axis from the first, unlocked position to the second, locked position. In addition, the distal portion 127 may include two opposing deflectable arms 129a and 129b, each having a tab 144a and 144b, respectively, in contact with an inner surface of the body 122, in the unlocked position. The inner surface of the body 122 may include first slots 140a and 140b and second slots 142a and 142b made along its opposite sides for receiving the tabs 144a, 144b therein. The first slots 140a, 140b and second slots 142a, 142b may be sized and shaped to receive the tabs 144a and 144b, respectively.

In the first, unlocked position, the tabs 144a, 144b may be positioned in the first slots 140a, 140b, respectively to retain the locking mechanism 124 in the unlocked position. When the locking mechanism 124 is moved to the second, locked position, the tabs 144a, 144b may be deflected out of the first slots 140a, 140b and then into the second slots 142a, 142b as the locking mechanism 124 is moved longitudinally relative to the body 122. In some instances, the tabs 144a, 144b may include an angled surface configured to engage a surface of the body 122 to deflect the arms 129a, 129b inward and thus disengage the tabs 144a, 144b from the first slots 140a, 140b as the locking mechanism 124 is moved to the locked position from the unlocked position.

The suture anchor 110 may be switched to the locked position by pushing the locking mechanism 124 into the cavity of body 122 as shown by the arrow in FIG. 7. In the locked position, as shown in FIG. 7, the distal portion 127 of the locking mechanism 124 may be pushed such that it may pass over the opening 126 and the tabs 144a and 144b may slide and lock into the second slots 142a and 142b, respectively, made in the inner surface of the body 122. Once inserted into the second slots 142a, 142b in the locked position, the tabs 144a, 144b may be precluded from disengaging from the second slots 142a, 142b. For example, the tabs 144a, 144b may be prevented from being moved back to the first slots 140a, 140b, and thus preventing unlocking the locking mechanism 124. Accordingly, the suture anchor 110 may be configured such that the locking mechanism 124 may be prevented from returning to the unlocked position once the locking mechanism 124 has been actuated to the locked position.

In the locked position, shown in FIG. 7, a suture (not shown) extending through the suture opening 126 may be clamped or pressed between a surface of the body 122 and a surface of the locking mechanism 124 to securely clamp the suture anchor 110 to the suture and prevent the suture from moving through the suture opening 126.

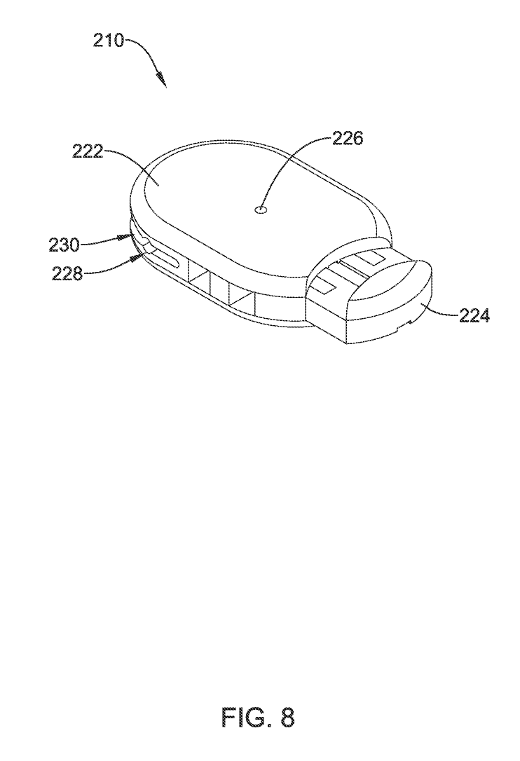

FIG. 8 is a perspective view of another exemplary suture anchor 210 that may also be used in conjunction with the percutaneous fixation assembly of FIG. 1. The suture anchor 210 may be similar to the suture anchor 110 in many respects. For example, the suture anchor 210 may include a body 222 and a locking mechanism 224 actuatable relative to the body 222 within a cavity of the body 222. The body 222 may include a suture opening 226 extending through the body 222 to receive a suture therethrough. The body 222 may also have an elongate channel 230 with an enlarged opening 228 in which the stylet 114 may be positioned when the suture anchor 210 is clipped onto or otherwise coupled to the stylet 114, similar to that described above regarding the suture anchor 110.

In contrast to the suture anchor 110 of FIGS. 4-7, the suture anchor 210 may have an oval shaped configuration. This configuration may be formed using similar materials as used for manufacturing of the suture anchor 110, as discussed previously.

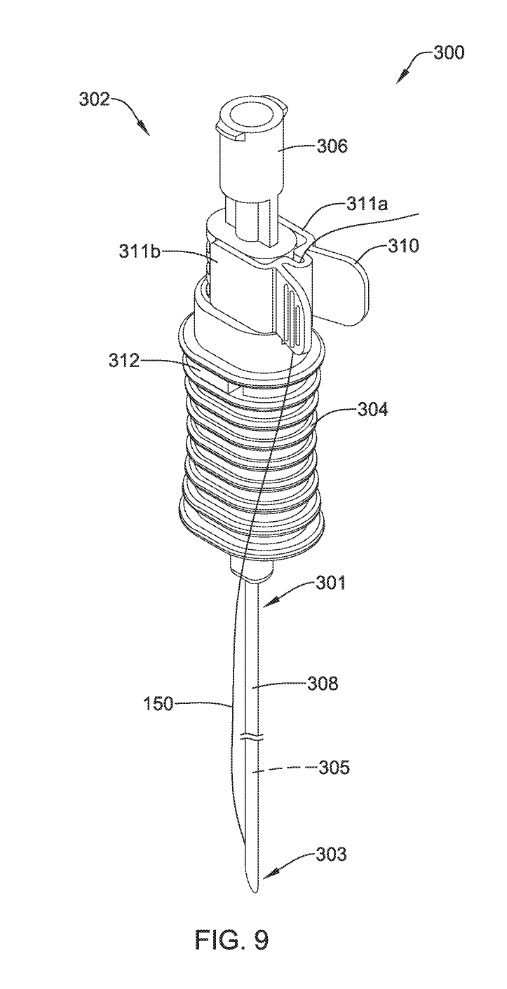

FIG. 9 is a perspective view of another exemplary percutaneous fixation assembly 300, in accordance with some embodiments of the present disclosure.

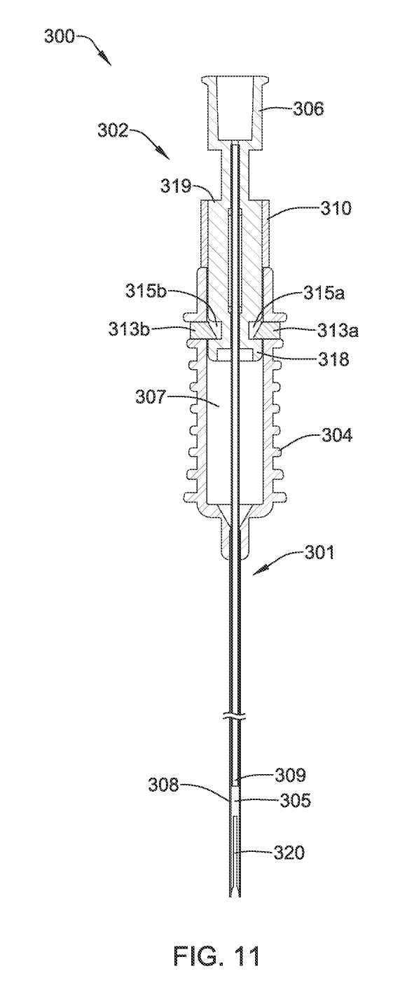

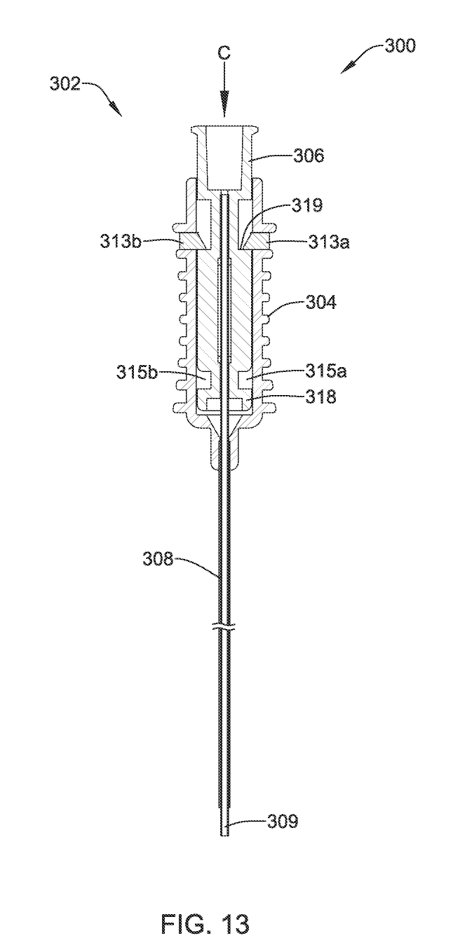

The fixation assembly 300 may include a hub assembly 302 having a needle hub 304 secured to a needle 308 and a stylet hub 306 secured to a stylet 309. The needle hub 304 may include an inner surface defining a bore 307 (see FIG. 11), such that a distal portion of the stylet hub 306 may extend into the bore 307 of the needle hub 304 and move longitudinally relative to the needle hub 104. The stylet hub 306 may be selectively actuatable relative to the needle hub 304 between a first position and a second position, which will be described in detail below. For example, the stylet hub 306 may be actuated relative to the needle hub 304 along a longitudinal axis of the elongate needle 308 and stylet 309 to move the stylet 309 through the elongate needle 308. For example, a push force may be applied in a distal direction along the longitudinal axis to actuate the stylet hub 306 from a first position (shown in FIG. 11) to a second position (shown in FIG. 13).

The elongate needle 308 of the fixation assembly 300 may have a proximal end 301, a distal end 303, and a lumen 305 extending between the proximal and distal ends 301 and 303. The proximal end 301 of the elongate needle 308 may be secured to the needle hub 304. In some embodiments, the distal end 303 of the elongate needle 308 may be introduced into a patient's body, while the proximal end 301 that is secured to the needle hub 304 may remain external to the patient's body. For example, the elongate needle 308 may pierce through the skin of the patient to be inserted into the stomach and/or gastric cavity of the patient during a medical procedure, such as a gastropexy procedure. Such an elongate needle 308 may have a sharp distal tip, and a length suitable for travelling a path from external skin to the internal gastric cavity of the patient. In addition, the elongate needle 308 may have an outer diameter suitable for penetrating through skin and to traverse through the flesh of the patient, such as to cross the wall of a desired organ (e.g., the stomach) minimizing or preventing any injury to the surrounding tissue. An exemplary elongate needle 308 may include a 12 gauge, 14 gauge, or 16 gauge needle, for example.

The stylet 309 of the fixation assembly 300 may be secured to the stylet hub 306 using various securing mechanism such as, but not limited to, molding, gluing, threading, snap-fitting, and so forth. As shown in FIG. 11, the stylet 309 may have a proximal end secured to the stylet hub 306 and a distal end extending into the lumen 305 of the needle 308. In some instances, the stylet 309 may be hollow and include a lumen which may be in fluid communication with a proximal port of the stylet hub 306. Such an arrangement of the stylet 309 within the needle lumen 305 may allow for passing fluid therethrough during a medical procedure. For example, in a gastropexy procedure, if the elongate needle 308 is introduced into the patient's stomach for placing a feeding tube, then a physician may need to confirm an accurate placement of the distal end 303 of the elongate needle 308 prior to deployment of a fastener from the needle 308. In such an instance, a syringe may be attached to the stylet hub 306 to aspirate air or other fluid from the patient's stomach through the lumen of the stylet 309. Observation of aspirated air or other fluid in the syringe may confirm the accurate positioning of the distal end 303 of the elongate needle 308 inside the stomach cavity. For example, the stomach may be insufflated with air or carbon dioxide during the procedure, which may be aspirated in the syringe to confirm accurate placement of the distal end 303 of the needle 308 inside the stomach cavity. Alternatively, a contrast media may be injected through a syringe attached to the stylet hub 306 through the lumen of the stylet 309 to confirm accurate placement of the needle 308 by visualizing the location of the injected contrast media using fluoroscopic imaging. Upon confirmation of accurate placement of the distal end 303 of the elongate needle 308 in the stomach, the physician may deploy the fastener from the elongate needle 308. Those skilled in the art will understand that any other suitable procedures may also be employed to confirm the placement of elongate needle 308 in the desired organ such as the stomach.

The fixation assembly 300 may also include a stop which may be configured to be removably coupled to the hub assembly 302 to prevent actuation of the stylet hub 306 from the first position to the second position. In some instances, as shown in FIG. 9, the stop may be a removable structure such as a clip 310 having a first clamping arm 311 a and a second clamping arm 311b. The clip 310 may be positionable about an external surface of the stylet hub 306. In particular, the first and second clamping arms 311a and 311b may be positioned on opposing sides of the stylet hub 306 proximal of the needle hub 304. The clip 310 may impede or prevent the stylet hub 306 from being actuated relative to the needle hub 304, thereby maintaining the stylet hub 306 in a first position. In the first position, the stylet 309 may remain inside the lumen 305 of the elongate needle 308.

The stylet 309 may be configured to deploy a fastener (not shown) that may be disposed within the lumen 305 of the elongate needle 308 distal of the stylet 309. In the first position, the fastener may remain inside the lumen 305 of the elongate needle 308 distal of the distal end of the stylet 309 disposed therein. The fastener may be secured to a suture 150, which may pass out of the elongate needle 308 through a slot 320 formed in a sidewall of the elongate needle 308 such that the suture 150 may extend along an exterior of the elongate needle 308 to the hub assembly 302 and/or a suture anchor, and pass through a suture opening in the suture anchor (e.g., pre-assembled with the suture anchor).

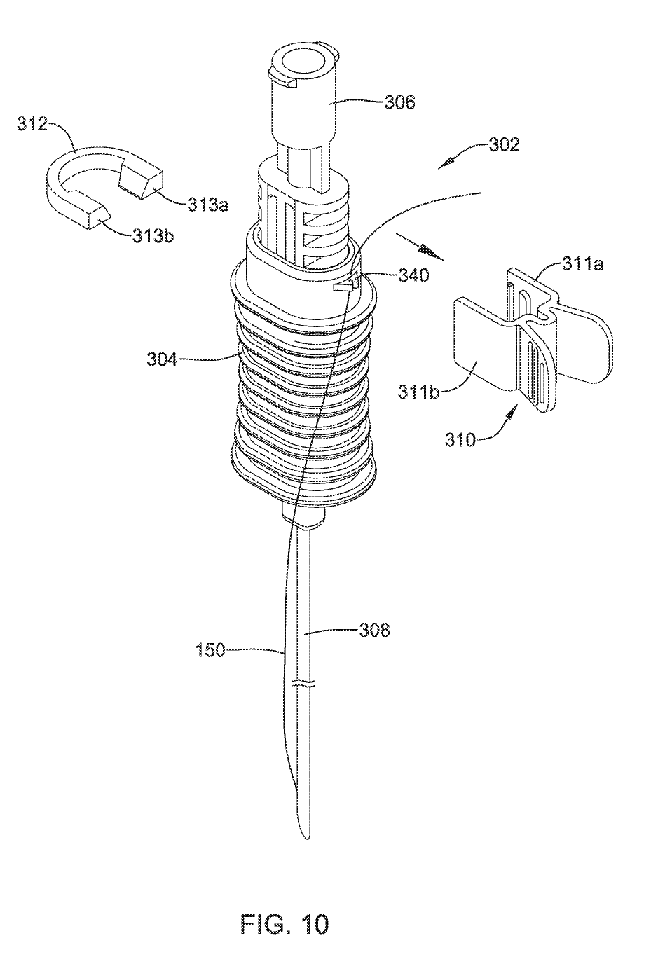

FIG. 10 is a perspective view showing various components of the percutaneous fixation assembly 300 of FIG. 9. In addition to the components discussed in FIG. 9 above, the fixation assembly 300 may include a retention mechanism configured to resist relative movement between the needle hub 304 and the stylet hub 306 unless a force greater than a threshold amount of force is applied. For example, the retention mechanism may include a clip, such as a C-shaped clip 312 having tabs 313a and 313b. The clip 312 may be similar to the clip 112 described with the embodiment of FIG. 1. The clip 312 may be wrapped around an external surface of the needle hub 304 such that the two tabs 313a and 313b may extend into the bore 307 (see FIG. 11) of the needle hub 304 from opposing sides of the bore 307. To this end, the needle hub 304 may include a channel or groove for receiving the C-shaped clip 312 and may include openings on opposite sides of the needle hub 304 for receiving the tabs 313a, 313b therein, such that the tabs 313a and 313b may pass therethrough to extend into the bore 307.

As shown in FIG. 11, a distal portion 318 of the stylet hub 306 may include a recess, such as grooves 315a and 315b formed on its external surface such that the tabs 313a and 313b of the C-shaped clip 312 may be positioned into the grooves 315a and 315b, and thereby may mechanically couple the stylet hub 306 and the needle hub 304 in the first position. In the first position, a proximal portion of the stylet hub 306 may include a groove 315 that may receive the tabs 313a and 313b in the second position.

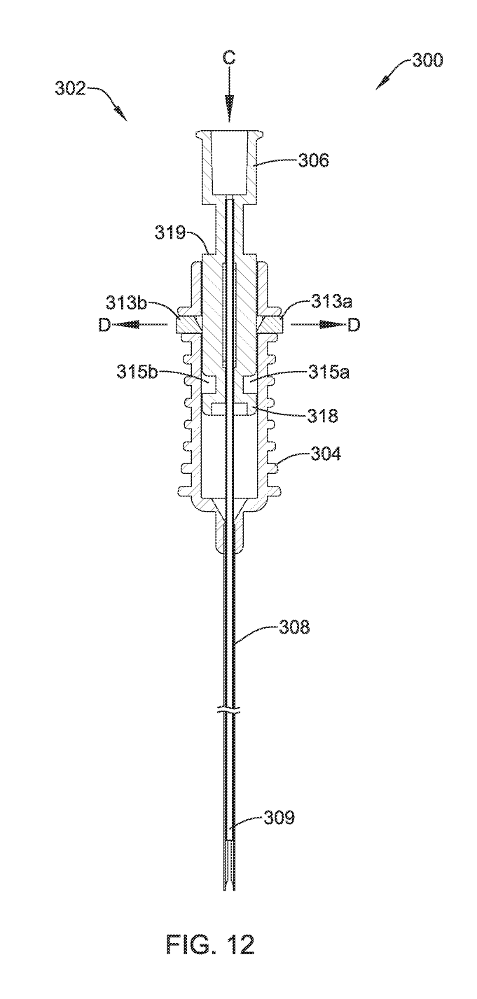

The stylet hub 306 may be pushed distally along a direction C (see arrow in FIG. 12), such that the tabs 313a and 313b may disengage with the grooves 315a and 315b, as shown in FIG. 12. Further pushing of the stylet hub 306 may deflect the tabs 313a and 313b away from one another along a direction D (see arrow in FIG. 12) to disengage from the recess or grooves 315a, 315b. The stylet hub 306 may be moved distally to the second position, shown in FIG. 13, until the tabs 313a and 313b are positioned above a groove or rim 319 of the stylet hub 306. The stylet hub 306 may then be in the second position, such that the engagement of tabs 313a and 313b with the rim 319 may impede or prevent actuation of the stylet hub 306 back to the first position from this second position.

Prior to being deployed from the needle 308, the suture 150 attached to the fastener may pass out of the elongate needle 308 through the slot 320 formed in a sidewall of the elongate needle 308 such that the suture 150 may extend along an exterior of the elongate needle 308 to the hub assembly 302 and/or a suture anchor. In some instances, the suture 150 may pass between the hub assembly 302 and the clip 310 while the clip 310 is attached to the hub assembly 302. In the second position, the distal end of the stylet 309, such as a blunt distal tip, may extend distally past the sharpened distal tip at the distal end 303 of the elongate needle 308 to deploy a fastener from the lumen 305 of the needle 308. In some instances, the handle assembly 302, such as the needle hub 304 may include a suture retainer 340 configured to receive the suture 150 therein. For example, the retainer 340 may include a notch, groove or other channel formed between tabs of the suture retainer 340 that the suture 150 can be clipped into. For example, the suture 150 may be snapped into a channel between the tabs of the suture retainer 340 to hold the suture 150 relative to the hub assembly 302.

In addition to the deployment of the fastener, the blunt distal tip of the stylet 309 may extend distally past the sharpened distal tip of the needle 308 to facilitate blunting of the elongate needle 308.

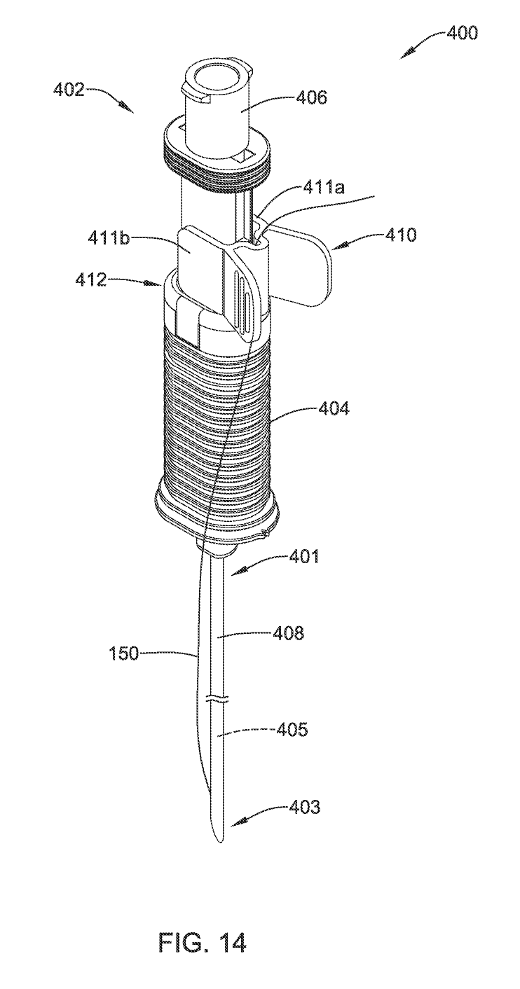

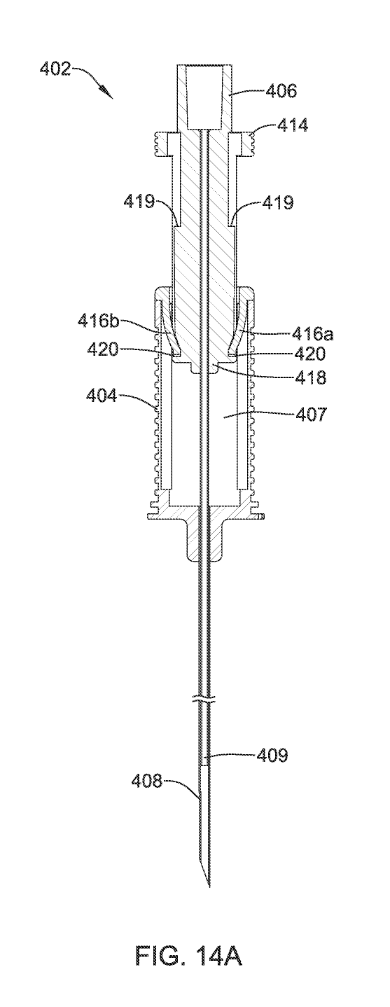

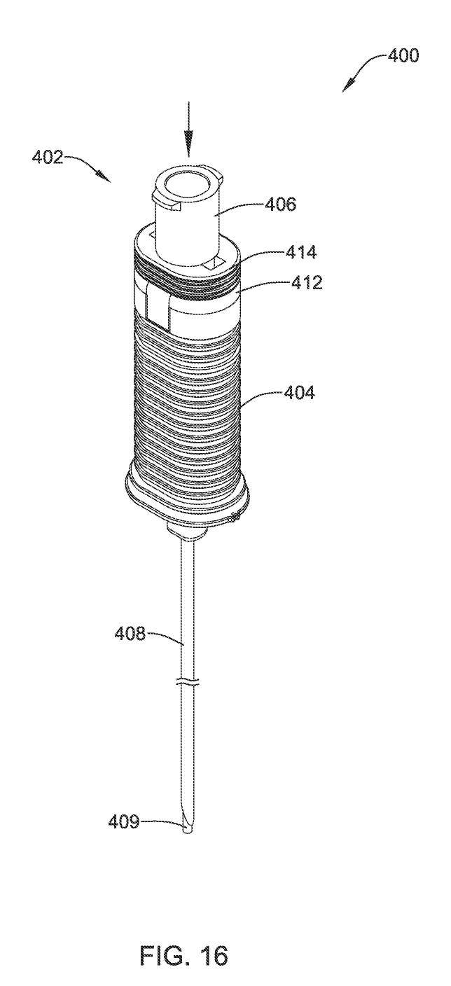

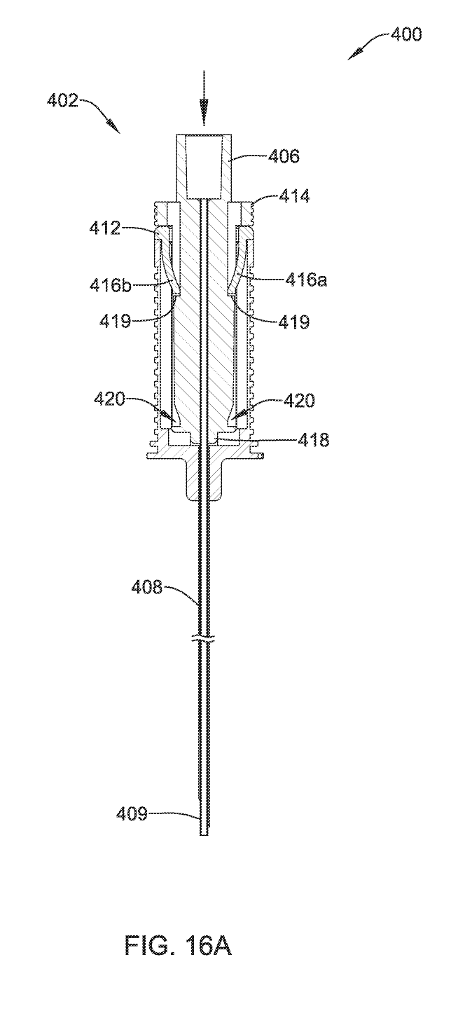

FIGS. 14 and 14A are perspective and longitudinal cross-sectional views of yet another exemplary percutaneous fixation assembly 400, in accordance with some embodiments of the present disclosure. The fixation assembly 400 may include a hub assembly 402 and an elongate needle 408 secured to the hub assembly 402. In particular, the hub assembly 402 includes a needle hub 404 and a stylet hub 406, such that a proximal end 401 of the elongate needle 408 may be secured to the needle hub 404 and a stylet 409 may be secured to the stylet hub 406 and extend through a lumen 405 of the needle 408. As shown in FIG. 14A, a distal portion 418 of the stylet hub 406 may be disposed within a bore 407 (see FIG. 14A) of the needle hub 404 and movable therein. The stylet hub 406 may be selectively actuatable relative to the needle hub 404 between a first position (shown in FIGS. 14 and 14A) and a second position (shown in FIGS. 16 and 16A), which will be discussed in greater detail below.

The stylet 409 may be configured to deploy a fastener (not shown) that may be disposed within the lumen 405 of the elongate needle 408 distal of the stylet 409. In the first position, the fastener may remain inside the lumen 405 of the elongate needle 408 distal of the distal end of the stylet 409 disposed therein. The fastener may be secured to a suture 150, which may pass out of the elongate needle, such as through a slot formed in a sidewall of the elongate needle 408, such that the suture 150 may extend along an exterior of the elongate needle 408 to the hub assembly 402 and/or a suture anchor, and pass through a suture opening in the suture anchor (e.g., pre-assembly with the suture anchor).

As shown in FIG. 14, the fixation assembly may further include a stop which may be configured to be removably coupled to the hub assembly 302 to prevent actuation of the stylet hub 406 from the first position to the second position. In some instances, as shown in FIG. 14, the stop may be a removable structure, such as a clip 410. The clip 410 may be removably coupleable to the hub assembly 402. In particular, the clip 410 may be wrapped about the stylet hub 406, which may impede or prevent the stylet hub 406 from being actuated relative to the needle hub 404. As a result, the stylet hub 406 may be maintained in the first position. In the first position, the stylet 409 (see FIG. 14A) may remain inside the lumen 405 of the elongate needle 408.

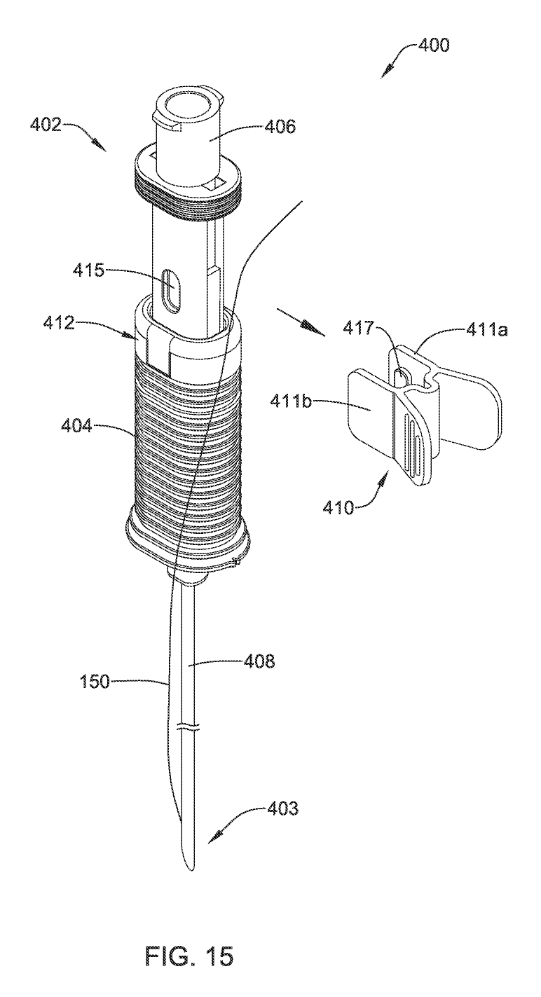

Structurally, the clip 410 may include a first clamping arm 411a and a second clamping arm 411b that may be positioned on opposing sides of the stylet hub 406 proximal of the needle hub 404. Further, the clip 410 may have a protrusion 417 (see FIG. 15) made on an inner surface of the first clamping arm 411a and/or the second clamping arm 411b, which may fit in an opening 415 (see FIG. 15) made in the stylet hub 406. In this manner, the clip 410 may be coupled to the stylet hub 406. Although not shown, the clip 410 may include a similar protrusion made on an inner surface of the second clamping arm 411b, if desired.

Prior to being deployed from the needle 408, the suture 150 attached to the fastener may pass out of the elongate needle 408 through a slot formed in a sidewall of the elongate needle 408 such that the suture 150 may extend along an exterior of the elongate needle 408 to the hub assembly 402 and/or a suture anchor. In some instances, the suture 150 may pass between the hub assembly 402 and the clip 410 while the clip 410 is attached to the hub assembly 402.

The fixation assembly 400 may further include a retention mechanism configured to resist relative movement between the needle hub 404 and the stylet hub 406 from the second, deployed position back to the first, undeployed position. For example, the retention mechanism may include one or more, or a plurality of tabs 416 configured to engage the stylet hub 406 to resist movement to the first position from the second position. For example, the tabs 416 may extend radially inward from the bore 407 of the needle hub 404 to engage a portion of the stylet hub 406 positioned therein.

In some instances, the tabs 416a, 416b may be formed as a unitary portion of a ring 412 that may be coupled to the needle hub 404. In other instances, the tabs 416a, 416b may be formed as a unitary portion of the needle hub 404, or the tabs 416a, 416b may be formed as separate components coupled to the needle hub 404.

The ring 412 may include an annular structure coupled to the top portion of the needle hub 404, for example. The profile of ring 412 may be the same as that of the needle hub 404, if desired. In some instances, the ring 412 may be made as an integral structure of the needle hub 404; however, the ring 412 may also be made as a separate structure which may then be coupled to the needle hub 404.

As shown in FIG. 14A, the ring 412 may include two tabs 416a and 416b disposed within the bore 407. A distal portion 418 of the stylet hub 406 may include a notches or recesses 420, such that the tabs 416a and 416b may engage within the notches or recesses 420 in the first position, thereby resisting the actuation of the stylet hub 406 relative to the needle hub 404.

Further, the stylet hub 406 may be pushed distally towards the needle hub 404 such as to actuate the stylet hub 406 to the second position. FIGS. 16-16A are perspective and longitudinal cross-sectional views, respectively, of the percutaneous fixation assembly 400 of FIG. 14 showing the stylet hub 406 in the second position. The stylet hub 406 may be actuated to the second position to deploy a fastener attached to the suture 150 from the needle 408 during a medical procedure. For example, the stylet hub 406 may be pushed along a direction E (see arrow), which may be along the longitudinal axis of the needle 408 and stylet 409, moving the proximal portion 414 of the stylet hub 406 toward the proximal end of the needle hub 404. For example, the stylet hub 406 may be actuated distally relative to the needle hub 404 such that the proximal portion 414 of the stylet hub 406 comes in contact with and/or faces the upper surface of the ring 412. The tabs 416a, 416b may be deflected radially outward, e.g., generally perpendicular to the longitudinal axis of the needle 408 and stylet 409 as the stylet hub 406 is moved distally toward the needle hub 404, thereby disengaging the tabs 416a and 416b from the notches or recesses 420. Further pushing the stylet hub 406 distally may engage the tabs 416a and 416b in a recess made on either side of the stylet hub 406 adjacent its proximal portion 414 such that the tabs 416a, 416b are positioned above the rim 419 of the stylet hub 406 in the second position. Engagement of the tabs 416a, 416b above the rim 419 may lock the hub assembly 400 in the second, deployed position, thus preventing the stylet hub 406 from moving back to the first position from the second position. In the second position in which a fastener may be deployed from the needle 408, the stylet 409 may travel distal past the elongate needle 408, thereby blunting the needle tip.

It is noted that in other embodiments, one or more, or a plurality of tabs may be formed with or coupled to the stylet hub 406 and be configured to engage one or more, or a plurality of notches or recesses in the needle hub 404 in a similar manner to retain the hub assembly 402 in a desired position, if desired.

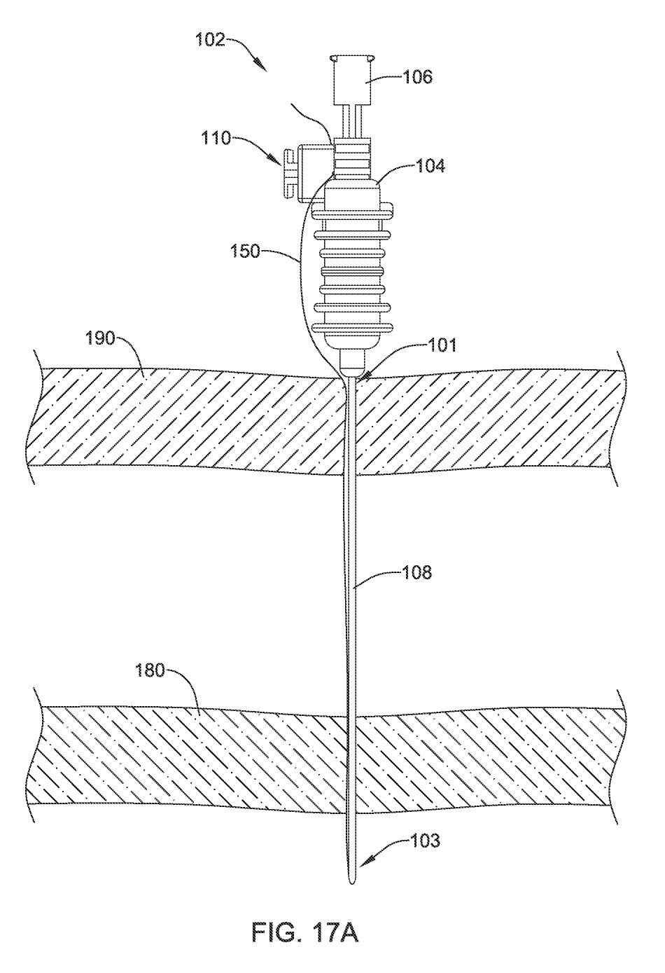

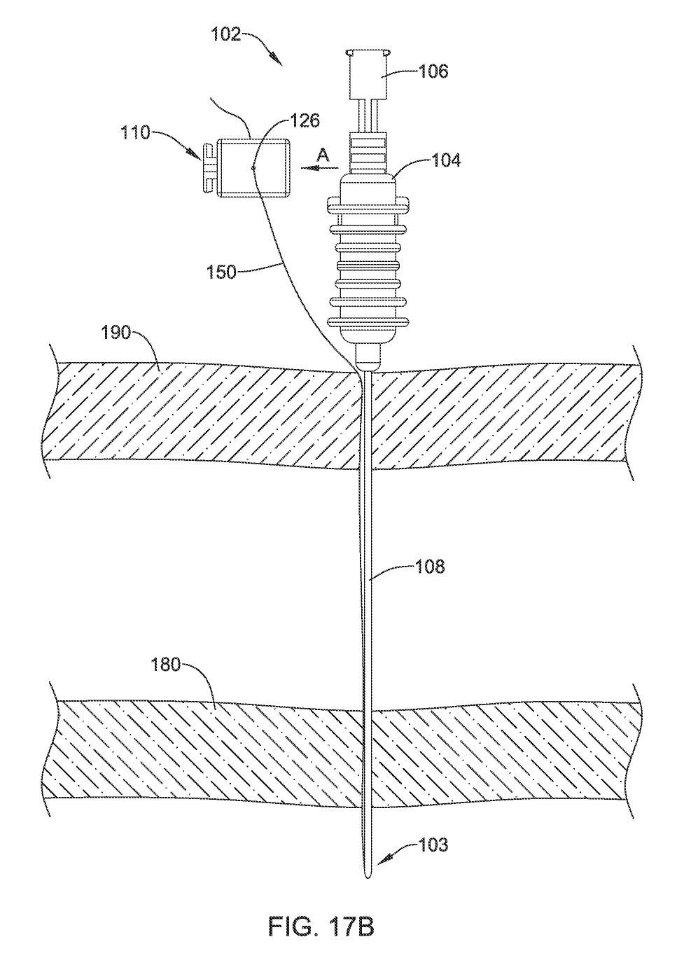

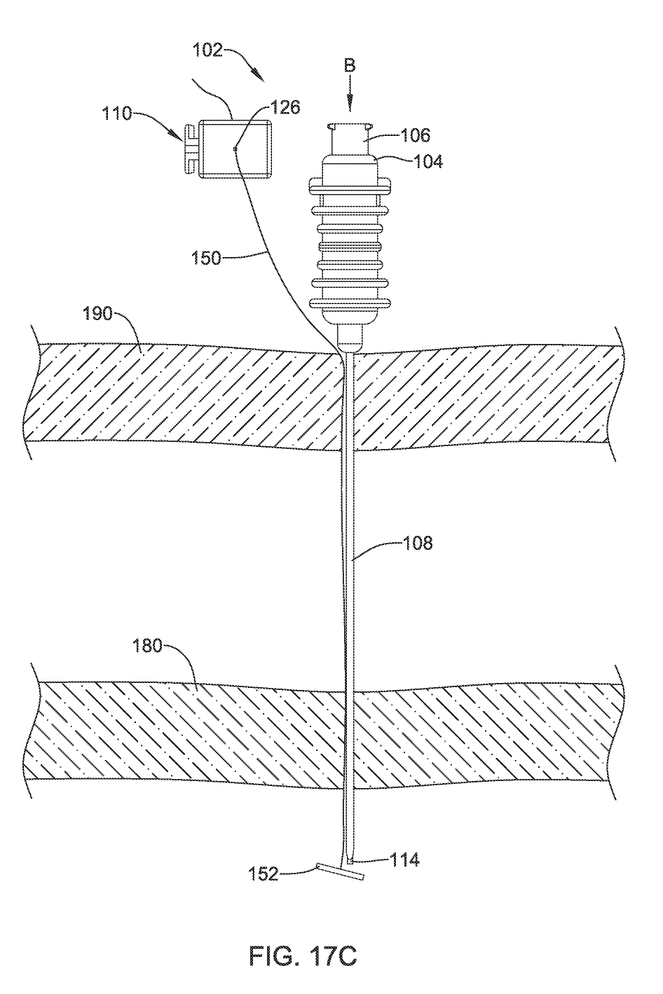

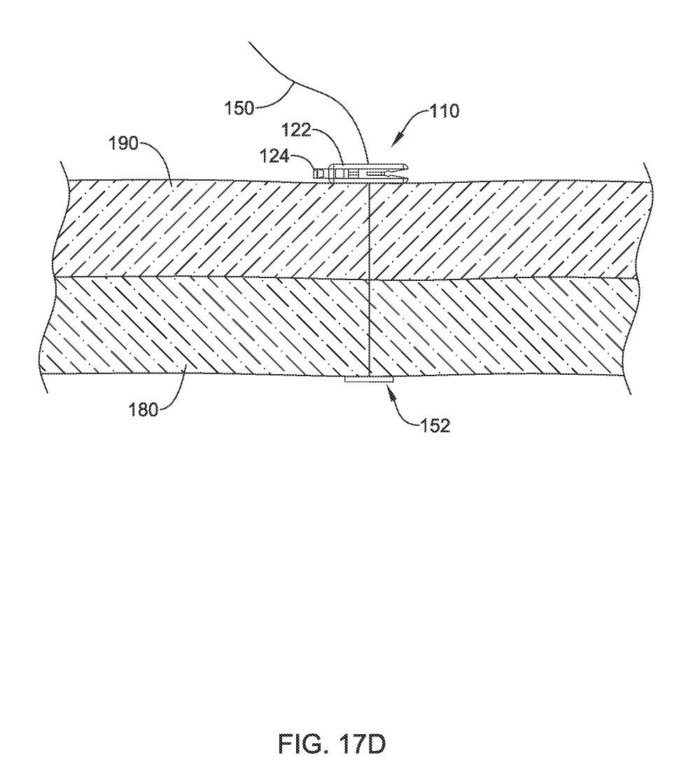

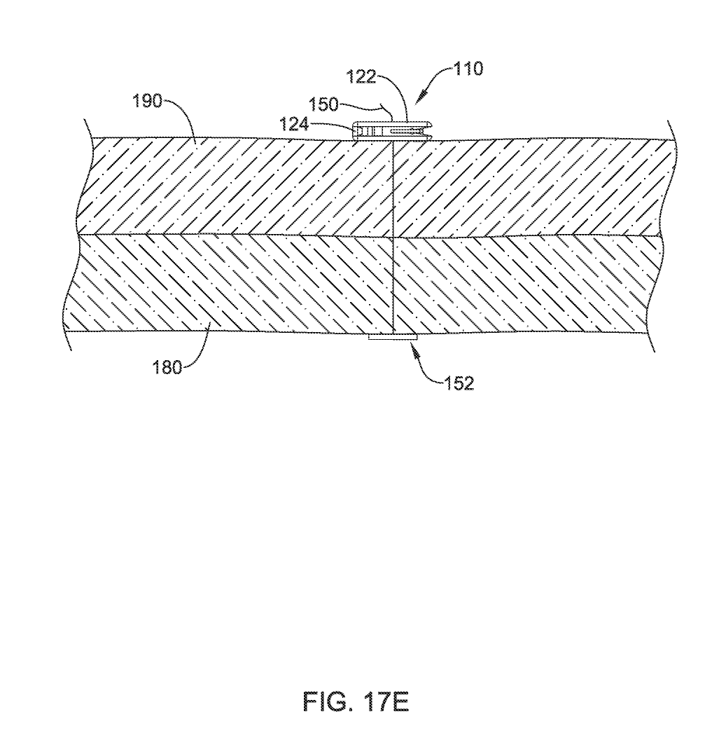

FIGS. 17A-17E shows various steps of an exemplary method of using the percutaneous fixation assembly 100 of FIG. 7. It is recognized, however, that additional embodiments of a percutaneous fixation assembly disclosed herein may be used in a similar way to deploy a suture in a medical procedure. In the exemplary method, the percutaneous fixation assembly 100 may be employed to secure the gastric wall 180 to the abdominal wall 190 such that a physician may subsequently place a feeding tube within the gastric cavity. Although the below discussed method may employ the fixation assembly 100 having similar components as that of the percutaneous fixation assembly 100 of FIG. 1, the percutaneous fixation assembly 100 may include any of the percutaneous fixation assemblies 300, 400 of FIGS. 9-16 as well.

During a percutaneous fixation procedure, such as a gastropexy procedure, the elongate needle 108 may be inserted into the patient's body to reach a desired anatomical location, such as the gastric cavity. To this end, the needle 408 may have a sharp tip, which may allow the needle 408 to pierce through the patient's skin. The needle 108 may have a lumen 105 within which a fastener 152 attached to a suture 150 may be initially positioned. The suture 150 coupled to the fastener 152 may extend from the fastener 152 out through the slot 120 in the sidewall of the needle 108 and extend along an exterior of the needle 108 up to the suture anchor 110, which is initially coupled to the hub assembly 102 to act as a stop to prevent inadvertent actuation of the stylet hub 106 relative to the needle hub 104 to the second position, and thus prevent premature deployment of the fastener 152. The suture 150 may pass through the suture opening 126 of the suture anchor 110 with the suture anchor 110 coupled to the handle assembly 102 in an unlocked position. Thus, the suture anchor 110 (in an unlocked position) may be pre-loaded on the suture 150 while the suture anchor 110 is coupled to the hub assembly 102. It may be noted that the suture 150 may run along the length of the needle 108, while the needle 108 is inserted into the patient's stomach. The suture anchor 110 may be positioned inside an opening in the stylet hub 106, which may prevent the actuation of stylet hub 106 relative to the needle hub 104 thereby maintaining the stylet hub 106 in a first position.

With the hub assembly 102 in the first position and the stop (e.g., the suture anchor 110) preventing inadvertent actuation of the hub assembly 102, the needle 108 may penetrate through the abdominal wall 190 and the gastric wall 180 such that the distal end 103 of the needle 108 is positioned in the patient's stomach.

Confirmation of accurate placement of the distal end 103 of the elongate needle 108 in the stomach prior to deployment of the fastener 152 may be achieved with a syringe coupled to the stylet hub 106 to aspirate air or other fluid from the patient's stomach, such as an insufflation fluid used to insufflate the stomach, through the lumen of the stylet 114. Observation of aspirated air or other fluid in the syringe may confirm the accurate positioning of the distal end 103 of the elongate needle 108 inside the stomach cavity. Alternatively, a contrast media may be injected through a syringe attached to the stylet hub 106 through the lumen of the stylet 114 to confirm accurate placement of the needle 108 by visualizing the location of the injected contrast media using fluoroscopic imaging. Upon confirmation of accurate placement of the distal end 103 of the elongate needle 108 in the stomach, the physician may deploy the fastener from the elongate needle 108.

Next, as shown in FIG. 17B, the suture anchor 110 may be removed from the hub assembly 102 to allow actuation of the stylet hub 106 relative to the needle hub 104 to the second position. For example, the suture anchor 110 may be removed along a direction A (see arrow) from the opening 111 of the stylet hub 106 to decouple the suture anchor 110 from the hub assembly 102. During this step, the suture 150 may continue to be positioned through the suture opening 126 of the suture anchor 110.

With the suture anchor 110, which was acting as a stop to prevent movement of the stylet hub 106 to the second position, removed, the hub assembly 102 may now be considered armed and able to be actuated to the second position to deploy the fastener 152. For example, after the suture anchor 110 (or other stop) is removed from the hub assembly 102, the stylet hub 106 may be actuated distally along a direction B (see arrow) into a second position, as shown in FIG. 17C. In the second position, the stylet hub 106 may be pushed distally and locked with the needle hub 104, such that the stylet 114 that was disposed within the lumen 105 of the elongate needle 108 may move past the distal end 103. As a result, the fastener 152 that was disposed within the lumen 105 distal of the stylet 114 may be pushed out of the lumen 105 by the stylet 114 and deployed within the gastric cavity. The fastener 152 may include a T-shaped anchor coupled to the suture 150, which may then be coupled to the suture anchor 110 external to the patient's body.

As a next step, as shown in FIG. 17D, the hub assembly 102 along with the needle 108 may be removed from the patient's stomach. The fastener 152 may be pulled proximally to draw the gastric wall 180 against the abdominal wall 190 and tightly secure the gastric wall 180 with the abdominal wall 190 using the suture anchor 110. For example, the suture anchor 110 may be advanced along the suture 110 until the suture anchor 110 is positioned against the skin of the patient. The suture 150 may be tensioned a sufficient amount to secure the gastric wall 180 against the abdominal wall 190. At this point, as shown in FIG. 17E, the suture 150 may be locked by locking the suture anchor 110. For example, the locking mechanism 124 may be actuated relative to the body 120 of the suture anchor 110 from the unlocked position to the locked position to lock the suture 150 to the suture anchor 110. At this stage, the extra length of the suture 150 may be cut.

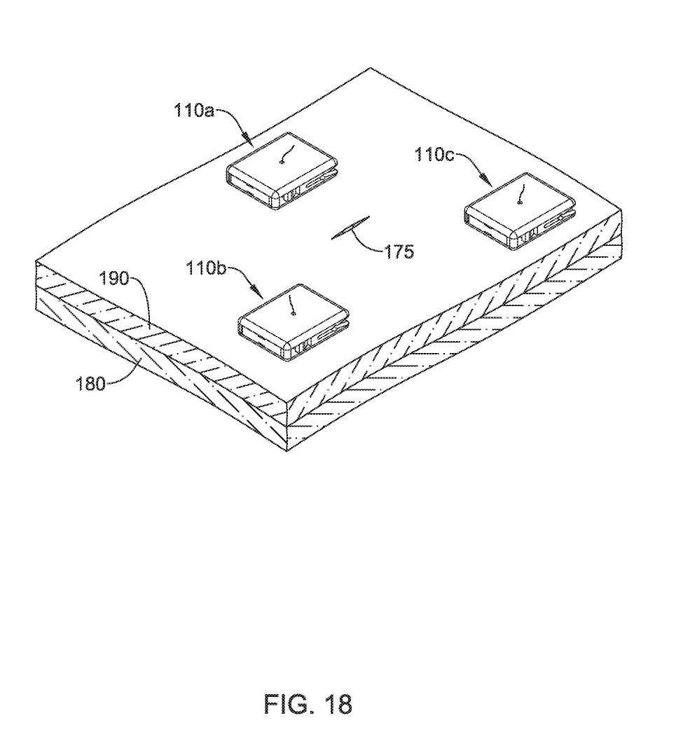

Any suitable number of fasteners 152 and corresponding sutures 150 may be deployed to tightly hold the two walls 190 and 180 during the gastropexy procedure. For example, as shown in FIG. 18, a procedure may include three suture anchors 110a-c that may hold three respective fasteners (not shown) deployed within the stomach cavity of a patient for placement of feeding tube during the gastropexy procedure. In the embodiment, three suture anchors 110a-c are positioned in a triangular configuration, such that a stoma and/or incision 175 may be made in middle of the triangle. The incision 175 may be employed to deliver the feeding tube inside the patient's stomach.

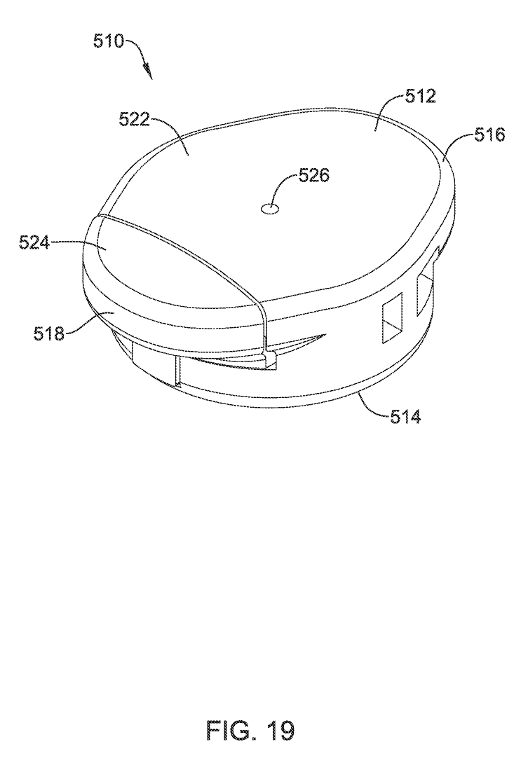

FIG. 19 is a perspective view of another exemplary suture anchor 510 for use with a percutaneous fixation assembly of this disclosure. The suture anchor 510 may be configured to securely lock onto a suture for use in a medical procedure.

The suture anchor 510 may be formed of a unitary, single component construction, or the suture anchor 510 may be formed of multiple components. For example, as shown, the suture anchor 510 may be formed as a two-component structure, which may include a body 522 and a locking mechanism 524 movable relative to the body 522. The locking mechanism 524 may be placed inside a cavity formed within the body 522. The locking mechanism 524 may be movable in the cavity of the body 522 between a first, unlocked position and a second, locked position. Although the suture anchor 510 is shown to be made as a two-component structure, the suture anchor 510 may be made as a unitary or monolithic structure, in which a locking mechanism portion is movable or deflectable relative to a body portion between an unlocked position and a locked position, if desired. The suture anchor 510 may also include a suture opening 526 configured to pass a suture therethrough.

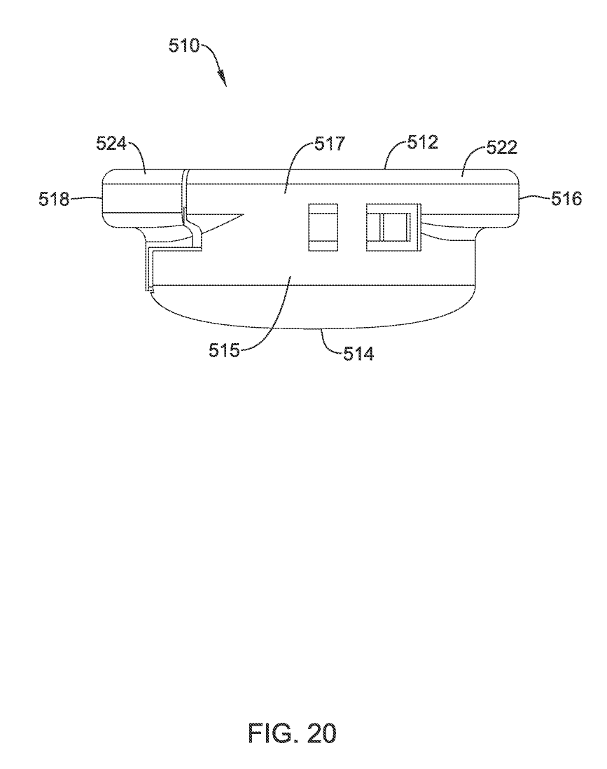

FIG. 20 is a side view of the suture anchor 510. The suture anchor 510 may have an upper surface 512 and an opposite lower surface 514 configured to be positioned against the skin of the patient. The upper surface 512 and/or the lower surface 514 may be planar or curved (e.g., convex, concave), as desired. The suture anchor 510 may have an upper portion 517, proximate the upper surface 512, which has a larger lateral profile than a lower portion 515 proximate the lower surface 514. For example, the upper portion 517 may extend outward further than the lower portion 515 in one or more, or in opposite directions, as viewed from the side to define wings 516, 518 overhanging the lower portion 515. In some instances the suture anchor 510 may be considered T-shaped when viewed from the side. The wings 516, 518 may provide additional surface area for the medical personnel to grip the suture anchor 510 (e.g., upper portion 517 including wings 516, 518), while reducing the surface area of the suture anchor 510 in contact with a patient's skin (e.g., lower surface 514 of lower portion 515). In the illustrated example, a first wing 516 may be formed in the body 522 and a second wing 518 may be formed in the locking mechanism 524, however, other configurations are contemplated. Furthermore, the configuration of the suture anchor 510 reduces the risk of the medical personnel from accidentally pinching the skin of the patient between the components of the suture anchor 510 by moving any pinch areas away from the skin of the patient.

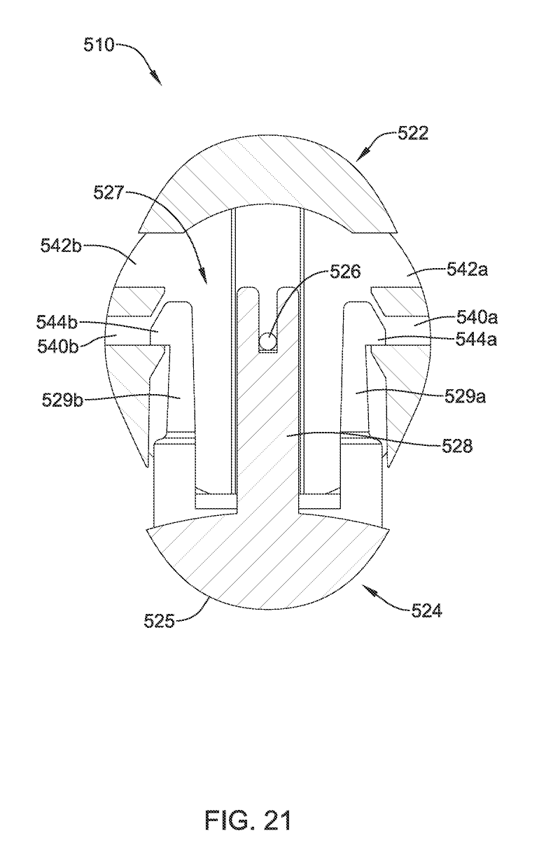

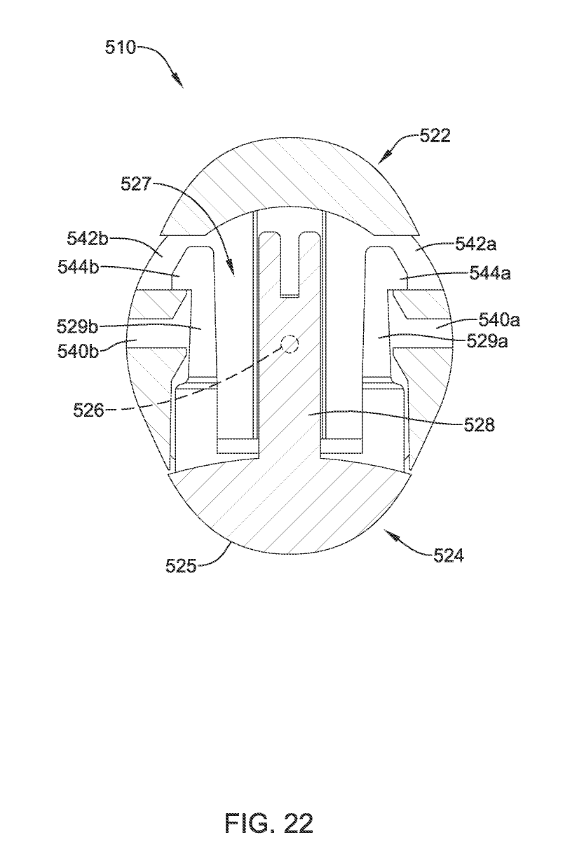

FIGS. 21-22 are cross-sectional top-views of the suture anchor 510 of FIG. 19 in unlocked and locked positions, respectively. As shown in FIG. 21 the locking mechanism 524 of the suture anchor 510 may include a proximal portion 525 and a distal portion 527. In the unlocked position, the proximal portion 525 may remain outside of the cavity of the body 522, whereas the distal portion 527 may be disposed within the cavity of the body 522. The distal portion 527 may be disposed substantially parallel along a longitudinal axis of the body 522 and actuatable along the longitudinal axis from the first, unlocked position to the second, locked position.

The distal portion 527 may include a center member or rail 528 movable along the longitudinal axis to interface with the body 522 to selectively clamp a suture therebetween. In addition, the distal portion 527 may include two opposing deflectable arms 529a and 529b, each having a tab 544a and 544b, respectively, in contact with an inner surface of the body 522, in the unlocked position. The inner surface of the body 522 may include first slots 540a and 540b and second slots 542a and 542b made along its opposite sides for receiving the tabs 544a, 544b therein. The first slots 540a, 540b and second slots 542a, 542b may be sized and shaped to receive the tabs 544a and 544b, respectively.

In the first, unlocked position, shown in FIG. 21, the tabs 544a, 544b may be positioned in the first slots 540a, 540b, respectively to retain the locking mechanism 524 in the unlocked position. When the locking mechanism 524 is moved to the second, locked position, shown in FIG. 22, the tabs 544a, 544b may be deflected out of the first slots 540a, 540b and then into the second slots 542a, 542b as the locking mechanism 524 is moved longitudinally relative to the body 522. In some instances, the tabs 544a, 544b may include an angled surface configured to engage a surface of the body 522 to deflect the arms 529a, 529b inward and thus disengage the tabs 544a, 544b from the first slots 540a, 540b as the locking mechanism 524 is moved to the locked position from the unlocked position.

The suture anchor 510 may be switched to the locked position by pushing the locking mechanism 524 into the cavity of the body 522. In the locked position, as shown in FIG. 22, the center member or rail 528 of the distal portion 527 of the locking mechanism 524 may be pushed such that it may pass over the opening 526 and the tabs 544a and 544b may slide and lock into the second slots 542a and 542b, respectively, made in the inner surface of the body 522. Once inserted into the second slots 542a, 542b in the locked position, the tabs 544a, 544b may be precluded from disengaging from the second slots 542a, 542b. For example, the tabs 544a, 544b may be prevented from being moved back to the first slots 540a, 540b, and thus preventing unlocking the locking mechanism 524. Accordingly, the suture anchor 510 may be configured such that the locking mechanism 524 may be prevented from returning to the unlocked position once the locking mechanism 524 has been actuated to the locked position.

In the locked position, shown in FIG. 22, a suture (not shown) extending through the suture opening 526 may be clamped or pressed between a surface of the body 522 and a surface of the locking mechanism 524 (e.g., a surface of the center member 528) to securely clamp the suture anchor 510 to the suture and prevent the suture from moving through the suture opening 526.

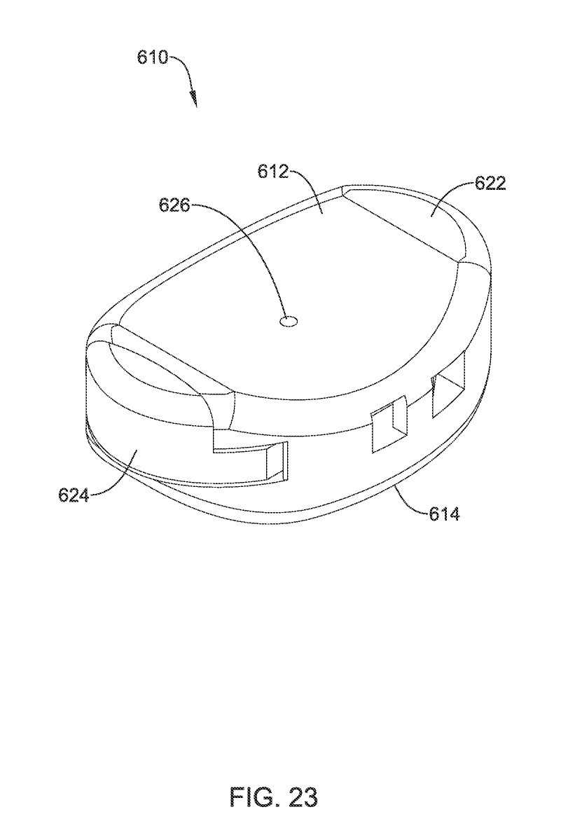

FIG. 23 is a perspective view of another exemplary suture anchor 610 for use with a percutaneous fixation assembly of this disclosure. The suture anchor 610 may be configured to securely lock onto a suture for use in a medical procedure. In some instances, the suture anchor 610 may be formed of a unitary, single component construction, or the suture anchor 610 may be formed of multiple components. As shown, the suture anchor 610 may be formed as a two-component structure, which may include a body 622 and a locking mechanism 624 movable relative to the body 622, similar to the suture anchors described above. The locking mechanism 624 may be movable in the cavity of the body 622 between a first, unlocked position and a second, locked position. The suture anchor 610 may also include a suture opening 626 configured to pass a suture therethrough. The suture opening 626 may be a through hole extending through the body 622 from a lower surface 614 to an upper surface 612 of the body 622, for example.

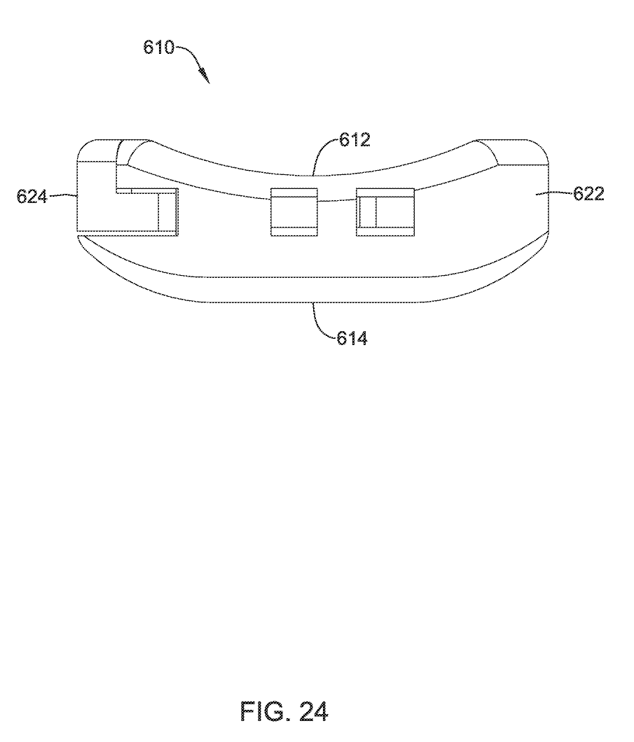

FIG. 24 is a side view of the suture anchor 610. The suture anchor 610 may have an upper surface 612 and an opposite lower surface 614 configured to be positioned against the skin of the patient. At least a portion of the upper surface 612 and/or the lower surface 614 may be curved (e.g., convex, concave), as desired. For example, at least a portion of the lower surface 614 may be convexly curved and at least a portion of the upper surface 612 may be concavely curved. For example, the lower surface 614 may include a central portion which may be flat or convexly curved, for example, and opposite side portions which may be convexly curved and/or angled upward. The configuration of the lower surface 614 (e.g., the convex curvature of the lower surface 614) may reduce the surface area of the suture anchor 610 in contact with a patient's skin while provide sufficient surface area for the medical personnel to grip the edges of the suture anchor 610. Furthermore, the configuration of the suture anchor 610 reduces the risk of the medical personnel from accidentally pinching the skin of the patient between the components of the suture anchor 610 by moving any pinch areas away from the skin of the patient.

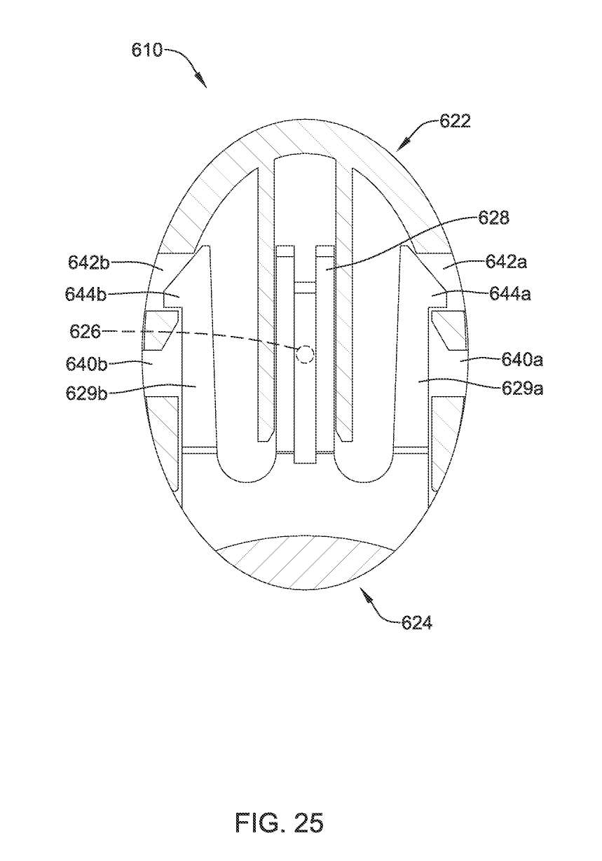

FIG. 25 is a cross-sectional top-view of the suture anchor 610 of FIG. 23 in a locked position. The suture anchor 610 may be constructed similar to the suture anchor 510, discussed above, such that the suture anchor 610 has similar locking features. For example, the locking mechanism 624 of the suture anchor 610 may include a center member or rail 628 movable along the longitudinal axis to interface with the body 622 to selectively clamp a suture therebetween. In addition, the locking mechanism 624 may include two opposing deflectable arms 629a and 629b, each having a tab 644a and 644b, respectively, positionable in first slots 640a and 640b of the body 622, in the unlocked position and positionable in second slots 642a and 642b of the body 622 in the locked position.

The tabs 644a, 644b may be positioned in the first slots 640a, 640b, respectively to retain the locking mechanism 524 in the unlocked position. When the locking mechanism 624 is moved to the second, locked position, the tabs 644a, 644b may be deflected out of the first slots 640a, 640b and then into the second slots 642a, 642b as the locking mechanism 624 is moved longitudinally relative to the body 622. In some instances, the tabs 644a, 644b may include an angled surface configured to engage a surface of the body 622 to deflect the arms 629a, 629b inward and thus disengage the tabs 644a, 644b from the first slots 640a, 640b as the locking mechanism 624 is moved to the locked position from the unlocked position. In the locked position, the center member or rail 628 of the locking mechanism 624 may be pushed such that it may pass over the opening 626 to securely clamp a suture between the center member 628 and the body 622 to prevent the suture from moving through the suture opening 626.

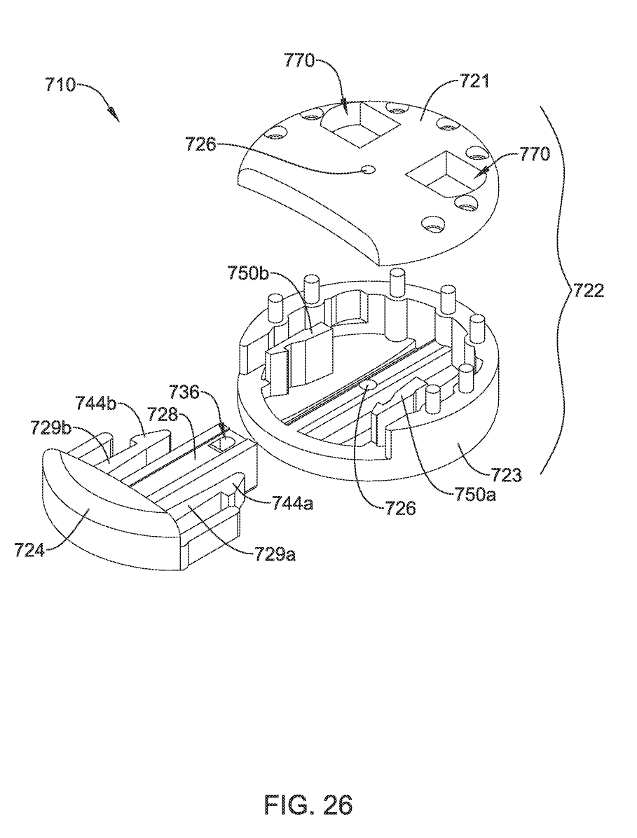

FIG. 26 is an exploded view of another exemplary suture anchor 710 for use with a percutaneous fixation assembly of this disclosure. The suture anchor 710 may be configured to securely lock onto a suture for use in a medical procedure.

The suture anchor 710 may include a body 722 and a locking mechanism 724 movable relative to the body 722. In some instances, the body 722 may be formed of multiple components, such as a first, upper member 721 and a second, lower member 723. The first and second members 721, 723 may be secured together in any desired way, such as with adhesive, heat staking, threaded fasteners, snap fit, or other securement mechanism. The locking mechanism 724 may be placed inside a cavity formed within the body 722 and movable therein between a first, unlocked position and a second, locked position. The suture anchor 710 may also include a suture opening 726 configured to pass a suture therethrough. The suture opening 726 may extend through the first member 721 and/or the second member 723 of the body 722, for example.

The locking mechanism 724 of the suture anchor 710 may include a center member or rail 728 movable along the longitudinal axis to interface with the body 722 to selectively clamp a suture therebetween. The center member or rail 728 may include a suture through hole 736 through which a suture may extend as the suture passes between the suture opening 726 through the first and second members 721, 723 of the body 722. Alternatively, the center member or rail 728 may be configured similar to other embodiments described herein, in which the suture may pass around an edge of the center member or rail 728, such as through an open channel extending into the center member or rail 728. Furthermore, it is noted that any one of the other embodiments described herein may include a suture through hole, similar to this embodiment. In addition, the locking mechanism 724 may include two opposing deflectable arms 729a and 729b, each having a tab 744a and 744b, respectively

The body 722 may include engagement features for engaging with the tabs 744a, 744b of the first and second arms 729a, 729b, in an unlocked position and/or a locked position. For instance, the body 722 may include a first wall 750a and a second wall 750b. The walls 750a, 750b may be formed as a portion of the first member 721 and/or the second member 723 of the body 722, for example. The walls 750a, 750b may include lips or stops for engagement with the tabs 744a, 744b of the arms 729a, 729b to prevent movement of the locking mechanism 724 relative to the body 722 in at least one direction.

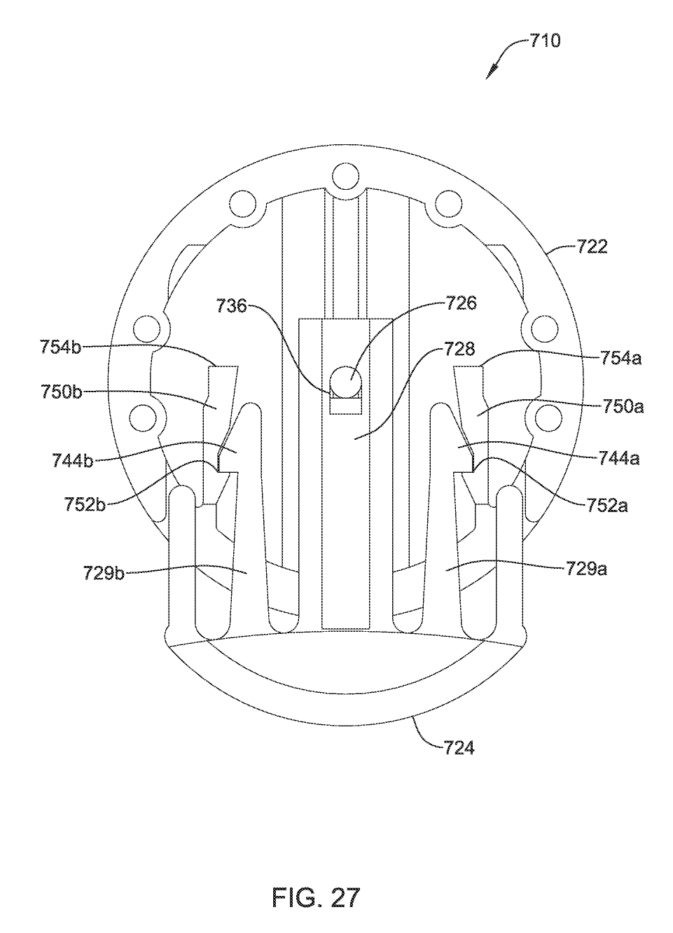

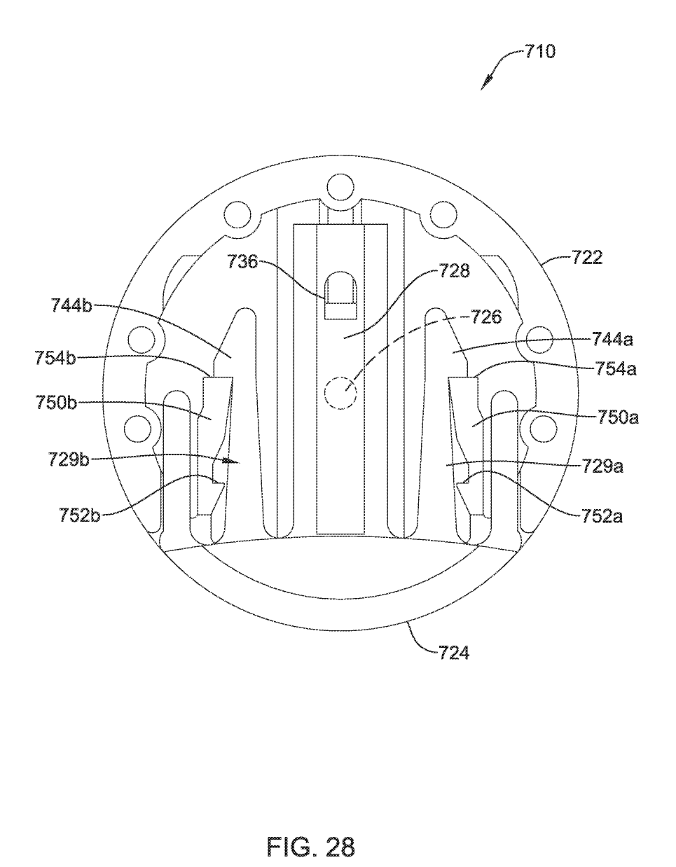

FIGS. 27-28 are cross-sectional top-views of the suture anchor 710 of FIG. 26 in unlocked and locked positions, respectively. In the first, unlocked position, shown in FIG. 27, a surface of the tabs 744a, 744b may abut a first stop 752a, 752b of the walls 750a, 750b, respectively to retain the locking mechanism 724 in the unlocked position. Engagement of the tabs 744a, 744b with the stops 752a, 752b may prevent the locking mechanism 724 from being removed from the body 722 while permitting the locking mechanism 724 to be moved from the unlocked position to the locked position.

When the locking mechanism 724 is moved to the second, locked position, shown in FIG. 28, the tabs 744a, 744b may be deflected as the tabs 744a, 744b move along the walls 750a, 750b as the locking mechanism 724 is moved longitudinally relative to the body 722 until the tabs 744a, 744b clear the second stop 754a, 754b. In some instances, the tabs 744a, 744b may include an angled surface configured to engage a surface of the walls 750a, 750b of the body 722 to deflect the arms 729a, 729b inward as the locking mechanism 724 is moved to the locked position from the unlocked position. Alternatively, the walls 750a, 750b may deflect as the locking mechanism 724 is moved to the locked position.

In the locked position, a surface of the tabs 744a and 744b may abut a second stop 754a, 754b, respectively, of the walls 750a, 750b of the body 722 to retain the locking mechanism 724 in the locked position. Once positioned in the locked position, the tabs 744a, 744b may be precluded from disengaging from the second stops 754a, 754b, and thus preventing unlocking the locking mechanism 724, without first actively disengaging the tabs 744a, 744b from the stops 754a, 754b. Accordingly, the suture anchor 710 may be configured such that the locking mechanism 724 may be prevented from returning to the unlocked position once the locking mechanism 724 has been actuated to the locked position, without actively manipulating the suture anchor 710 to disengage the arms 729a, 729b of the locking mechanism 724 from the walls 750a, 750 of the body 722.

In the locked position, as shown in FIG. 28, the center member or rail 728 of the locking mechanism 724 may be pushed such that it may pass over the opening 726 to move the through hole 736 out of alignment with the opening 726. In the locked position, a suture (not shown) extending through the suture opening 726 of the body 722 and the suture through hole 736 of the locking mechanism 724 may be clamped or pressed between a surface of the body 722 and a surface of the locking mechanism 724 (e.g., a surface of the center member 728) to securely clamp the suture anchor 710 to the suture and prevent the suture from moving through the suture openings 726, 736.

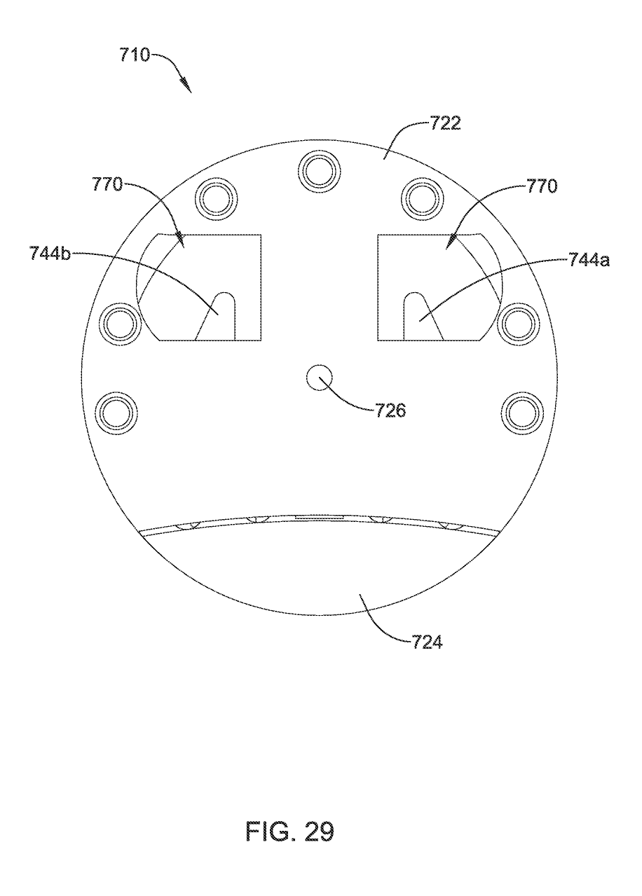

As shown in FIG. 29, the suture anchor 710 may include a means for selectively releasing engagement of the arms 729a, 729b (e.g., the tabs 744a, 744b) from the walls 750a, 750b of the body 722 to permit the locking mechanism 724 to be moved from the locked position back to the unlocked position if it is desired to remove the suture anchor 710, adjust the position of the suture anchor 710, adjust the tension of the suture, or otherwise manipulate the suture anchor 710. For example, the body 722 may include one or more, or a plurality of openings or windows 770 opening into an interior of the body 722. The windows 770 may be located on the upper surface of the body 722 (e.g., an upper surface of the first member 721), or at a different location of the body 722 accessible to medical personnel. A portion of the arms 729a, 729b (e.g., the tabs 744a, 744b) and/or the walls 750a, 750b may be visible through the windows 770.

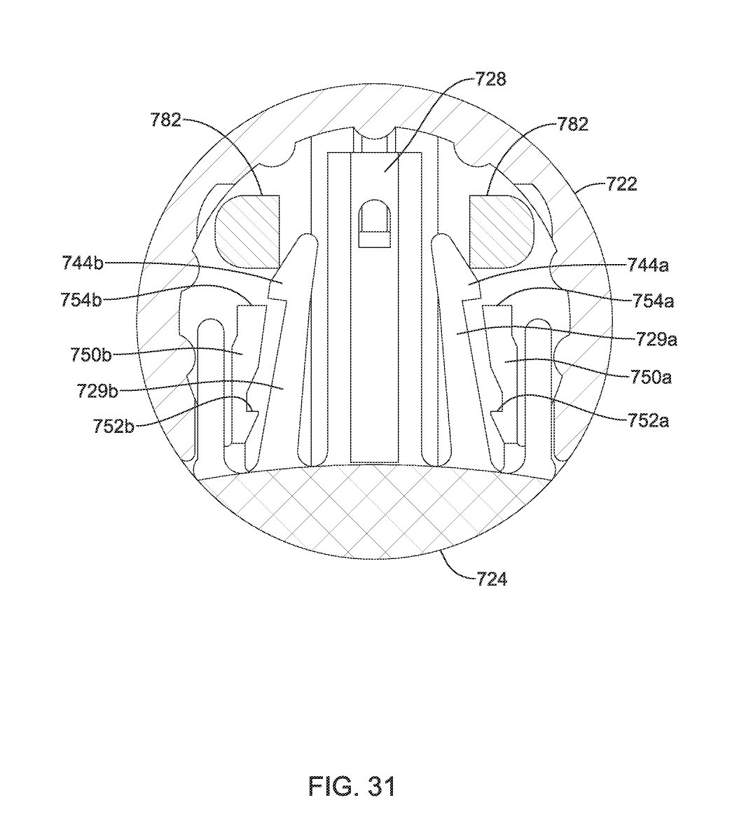

If it is desired to unlock the locking mechanism 724 from the locked position, an instrument may be used to disengage the arms 729a, 729b from the stops 754a, 754b of the walls 750a, 750b. For example, an instrument may be used to deflect the arms 729a, 729b to disengage the tabs 744a, 744b from the stops 754a, 754b and thus allow the locking mechanism 724 to move back to the unlocked position from the locked position. Alternatively, an instrument may be used to deflect the walls 750a, 750b to disengage the tabs 744a, 744b from the stops 754a, 754b and thus allow the locking mechanism 724 to move back to the unlocked position from the locked position. For example, a hemostat 780 or other instrument, may be inserted into the windows 770 to engage the tabs 744a, 744b of the arms 729a, 729b to deflect the arms 729a, 729b (or engage the walls 750a, 750b to deflect the walls 750a, 750b). For instance, as shown in FIG. 31, the clamping jaws 782 of the hemostat 780 may be inserted into the windows 770 to engage the tabs 744a, 744b to deflect the arms 729a, 729b toward each other and thus disengage the tabs 744a, 744b from the stops 754a, 754b. It is recognized that other mechanisms may be employed to disengage the arms 729a, 729b from the stops 754a, 754b of the walls 750a, 750b, if desired. For example, an integral portion of the walls 750a, 750b or arms 729a, 729b, or other member, may be accessible for a user to manipulate to disengage the arms 729a, 729b from the tops 754a, 754b, if desired. Once deflected, the locking mechanism 724 may be moved back to the unlocked position to remove or reposition the suture anchor 710, if desired.

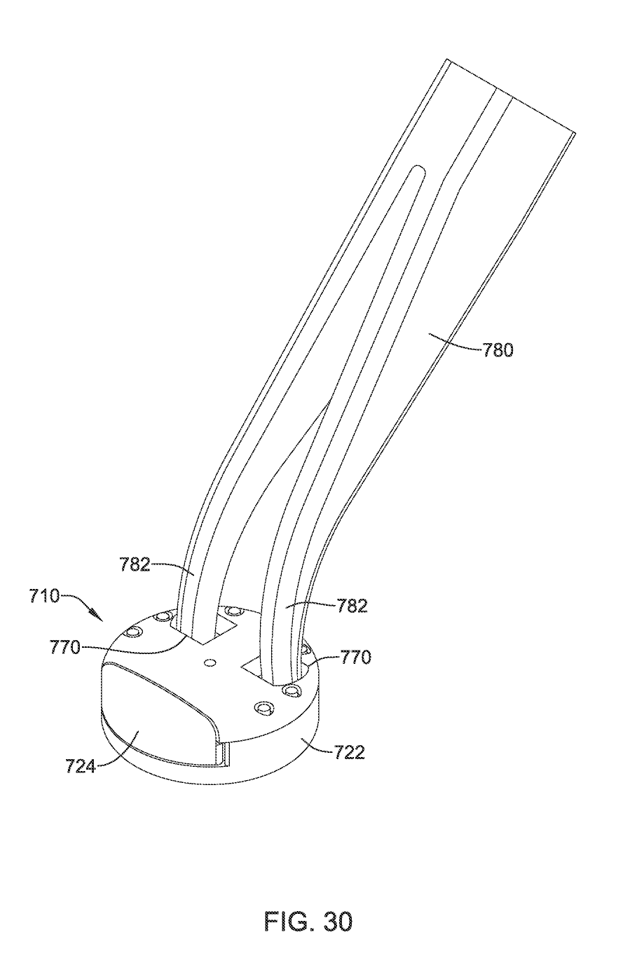

Although described with the suture anchor 710 of FIGS. 26-31, such a unlocking mechanism may be incorporated with any of the other suture anchor embodiments described herein to permit selective unlocking of the locking mechanism from the body of the suture anchor to remove, reposition, re-tension, or otherwise manipulate the suture anchor after it has been locked.

It should be understood that this disclosure is, in many respects, only illustrative. Changes may be made in details, particularly in matters of shape, size, and arrangement of steps, without exceeding the scope of the disclosure. This may include, to the extent that it is appropriate, the use of any of the features of one exemplary embodiment in other embodiments. The disclosure's scope is, of course, defined in the language in which the appended claims are expressed.

### Additional Examples

In a first example, a percutaneous fixation assembly comprises a hub assembly including a needle hub and a stylet hub selectively actuatable relative to the needle hub between a first position and a second position; an elongate needle having a proximal end, a distal end, and a lumen extending therethrough, the proximal end of the elongate needle secured to the needle hub; a stylet secured to the stylet hub and extending into the lumen of the elongate needle; and a stop removably coupleable to the hub assembly when the stylet hub is in the first position to prevent actuation of the stylet hub to the second position. The stop is removable from the hub assembly to allow actuation of the stylet hub to the second position.

Alternatively or additionally, in a second example, the stop is a suture anchor.

Alternatively or additionally, in a third example, the stylet hub includes an opening and the suture anchor is positionable in the opening of the stylet hub in the first position.

Alternatively or additionally, in a fourth example, the needle hub includes a tab extendable into the opening of the stylet hub in the second position.

Alternatively or additionally, in a fifth example, the tab prevents actuation of the stylet hub to the first position from the second position.

Alternatively or additionally, in a sixth example, the percutaneous fixation assembly of any of examples 1-5 includes a fastener and a suture extending from the fastener, the fastener being positionable in the lumen of the elongate needle distal of the stylet and the suture passing through an opening in the suture anchor.

Alternatively or additionally, in a seventh example, the suture anchor includes an elongate channel, wherein the stylet is positioned in the elongate channel of the suture anchor when the suture anchor is coupled to the hub assembly.

Alternatively or additionally, in an eight example, the suture anchor includes an opening for receiving a suture therethrough, the opening extending perpendicular to the elongate channel.

Alternatively or additionally, in a ninth example, the suture anchor includes a body and a locking mechanism moveable relative to the body from an unlocked position to a locked position.

Alternatively or additionally, in a tenth example, the body of the suture anchor includes an elongate channel and the stylet is positioned in the elongate channel of the suture anchor when the suture anchor is coupled to the hub assembly such that the locking mechanism is prevented from moving to the locked position from the unlocked position.

Alternatively or additionally, in an eleventh example, the stop is a clip having first and second clamping arms positionable on opposing sides of the stylet hub proximal of the needle hub.

Alternatively or additionally, in a twelfth example, the needle hub includes an inner surface defining a bore and a distal portion of the stylet hub extends into the bore of the needle hub, and wherein the needle hub includes a deflectable tab extending inward from the inner surface into the bore, the tab configured to engage a notch in the needle hub in the second position to prevent movement of the stylet hub to the first position from the second position.

Furthermore, a thirteenth example includes a method of manipulating a percutaneous fixation assembly, including the steps of: providing a percutaneous needle assembly, the percutaneous needle assembly including a hub assembly including a needle hub and a stylet hub selectively actuatable relative to the needle hub, an elongate needle having a proximal end, a distal end, and a lumen extending therethrough, the proximal end of the elongate needle secured to the needle hub, a stylet secured to the stylet hub and extending into the lumen of the elongate needle, and a suture anchor removably coupled to the hub assembly with the stylet hub positioned in a first position, the suture anchor preventing actuation of the stylet hub to a second position; removing the suture anchor from the hub assembly to allow actuation of the stylet hub to the second position; and actuating the stylet hub to the second position after removing the suture anchor from the hub assembly.

Alternatively or additionally, in a fourteenth example, the suture anchor is coupled to a suture when coupled to the hub assembly.

Alternatively or additionally, in a fifteenth example, the suture anchor is positioned in an opening of the stylet hub when removably coupled to the hub assembly.

What is claimed is:

1. A medical device assembly, comprising:
a hub assembly including a needle hub and a stylet hub selectively actuatable relative to the needle hub between a first position and a second position;
an elongate needle having a proximal end, a distal end, and a lumen extending therethrough, the proximal end of the elongate needle secured to the needle hub;
a stylet secured to the stylet hub and extending into the lumen of the elongate needle; and
a stop removably coupled to the hub assembly when the stylet hub is in the first position to prevent actuation of the stylet hub to the second position;
wherein the stop is removable from the hub assembly to allow actuation of the stylet hub to the second position.

2. The medical device assembly of claim 1, wherein the stop is a suture anchor.

3. The medical device assembly of claim 2, wherein the stylet hub includes an opening and the suture anchor is positionable in the opening of the stylet hub in the first position.

4. The medical device assembly of claim 3, wherein the needle hub includes a tab extendable into the opening of the stylet hub in the second position.

5. The medical device assembly of claim 4, wherein the tab prevents actuation of the stylet hub to the first position from the second position.

6. The medical device assembly of claim 2, further comprising a fastener and a suture extending from the fastener, the fastener positionable in the lumen of the elongate needle distal of the stylet and the suture passing through an opening in the suture anchor.

7. The medical device assembly of claim 2, wherein the suture anchor includes an elongate channel, wherein the stylet is positioned in the elongate channel of the suture anchor when the suture anchor is coupled to the hub assembly.

8. The medical device assembly of claim 7, wherein the suture anchor includes an opening for receiving a suture therethrough, the opening extending perpendicular to the elongate channel.

9. The medical device assembly of claim 2, wherein the suture anchor includes a body and a locking mechanism moveable relative to the body from an unlocked position to a locked position.

10. The medical device assembly of claim 9, wherein the body of the suture anchor includes an elongate channel and the stylet is positioned in the elongate channel of the suture anchor when the suture anchor is coupled to the hub assembly such that the locking mechanism is prevented from moving to the locked position from the unlocked position.

11. The medical device assembly of claim 1, wherein the stop is a clip having first and second clamping arms positionable on opposing sides of the stylet hub proximal of the needle hub.

12. The medical device assembly of claim 11, wherein the needle hub includes an inner surface defining a bore and a distal portion of the stylet hub extends into the bore of the needle hub, and wherein the needle hub includes a deflectable tab extending inward from the inner surface into the bore, the tab configured to engage a notch in the needle hub in the second position to prevent movement of the stylet hub to the first position from the second position.

13. A medical device assembly, comprising:
a hub assembly including a needle hub and a stylet hub selectively actuatable relative to the needle hub between a first position and a second position;
an elongate needle having a proximal end, a distal end, and a lumen extending therethrough, the proximal end of the elongate needle secured to the needle hub;
a stylet secured to the stylet hub and extending into the lumen of the elongate needle;
a suture anchor removably coupled to the hub assembly when the stylet hub is in the first position to prevent actuation of the stylet hub to the second position; and
a fastener and a suture extending from the fastener, the fastener positionable in the lumen of the elongate needle distal of the stylet and the suture passing through an opening in the suture anchor;
wherein the suture anchor is removable from the hub assembly to allow actuation of the stylet hub to the second position.

14. The medical device assembly of claim 13, wherein the suture anchor includes an elongate channel, wherein the stylet is positioned in the elongate channel of the suture anchor when the suture anchor is coupled to the hub assembly.

15. The medical device assembly of claim 14, wherein the suture anchor includes a body and a locking mechanism moveable relative to the body from an unlocked position to a locked position.

16. The medical device assembly of claim 15, wherein the body of the suture anchor includes the elongate channel with the stylet positioned therein when the suture anchor is coupled to the hub assembly such that the locking mechanism is prevented from moving to the locked position from the unlocked position.

17. The medical device assembly of claim 13, wherein:
the stylet hub includes an opening and the suture anchor is positionable in the opening of the stylet hub in the first position; and
the needle hub includes a tab extendable into the opening of the stylet hub in the second position, the tab preventing actuation of the stylet hub to the first position from the second position when extended into the opening of the stylet hub.

18. A method of manipulating a fixation assembly, comprising:
providing a percutaneous needle assembly, the percutaneous needle assembly including:
a hub assembly including a needle hub and a stylet hub selectively actuatable relative to the needle hub;
an elongate needle having a proximal end, a distal end, and a lumen extending therethrough, the proximal end of the elongate needle secured to the needle hub;
a stylet secured to the stylet hub and extending into the lumen of the elongate needle; and
a suture anchor removably coupled to the hub assembly with the stylet hub positioned in a first position, the suture anchor preventing actuation of the stylet hub to a second position;
removing the suture anchor from the hub assembly to allow actuation of the stylet hub to the second position; and
actuating the stylet hub to the second position after removing the suture anchor from the hub assembly.

19. The method of claim 18, wherein the suture anchor is coupled to a suture when coupled to the hub assembly.

20. The method of claim 18, wherein the suture anchor is positioned in an opening of the stylet hub when removably coupled to the hub assembly.
